(19) United States
(12) Patent Application Publication
(10) Pub. No.: US 2004/0127895 A1
(43) Pub. Date: Jul. 1, 2004

(54) Electromagnetic treatment of tissues and cells

(76) Inventors: Stephen T. Flock, Arvada, CO (US); Kevin S. Marchitto, Golden, CO (US)

Correspondence Address: Benjamin Aaron Adler, ADLER & ASSOCIATES, 8011 Candle Lane, Houston, TX, 77071, US

(21) Appl. No.: 10/736,133

(22) Filed: Dec. 15, 2003

Related U.S. Application Data
(63) Continuation-in-part of application No. 10/441,341, filed on May 20, 2003.
(60) Provisional application No. 60/381,948, filed on May 20, 2002.

(51) Int. Cl. A61B 18/18; A61F 2/00

(52) U.S. Cl. 606/41; 607/100; 607/103

(57) ABSTRACT

Provided herein are methods, devices and compositions to conductively or to inductively heat substrates using electromagnetic energy. Also provided are methods, devices and compositions to inductively alter or treat a substrate by inducing a conformational change in the compositions and/or the substrates. The compositions provided comprise an energy absorbing species to transduce heat to the substrates to effect such alterations or treatments.

### CROSS-REFERENCE TO RELATED APPLICATIONS

This application is a continuation-in-part of non-provisional U.S. Ser. No. 10/441,341, filed May 20, 2003, which claims benefit of provisional application U.S. Ser. No. 60/381,948, filed May 20, 2002, now abandoned.

### BACKGROUND OF THE INVENTION

1. Field of the Invention

The present invention relates generally to the fields of biomedical engineering, biochemistry and medical treatment and surgical procedures. More specifically, the present invention provides methods, devices and compositions for inducing changes in biomolecules and bioactive molecules useful for accelerating or enabling certain reactions, fixing or fusing tissues and implants, dressing, sealing or closing a wound and delivery of active agents to tissues.

2. Description of the Related Art

Historically, wound dressings consist of some type of bandage or adhesive. More recently, wound sealing methods whereby energy is directed to the tissue have been tested and occasionally are used clinically. Traditional techniques of managing the wound include cleansing and debriding, treating with antibiotics and applying a dressing. Modern wound care products often seek to provide moisture, pH balance and nutrition in an effort to improve the potential for healing. The healing process may also complicate the status of the patient through formation of scar tissue. This scarring helps to close the wound, but its formation is accompanied by contraction and buildup of tissue which can lead to a loss in flexibility at the wound site and, in severe cases, may result in loss of mobility to the patient.

Commercial electrosurgery and electrocautery devices commonly are used for sealing internal wounds, such as those arising through surgical intervention. Inventions for sealing vessels using other forms of electromagnetic energy have been published. U.S. Pat. No. 6,033,401 describes a device to deliver adhesive and apply microwave energy to effect sealing of a vessel. U.S. Pat. No. 6,179,834 discloses a vascular sealing device to provide a clamping force, while radiofrequency energy is applied, until a particular temperature or impedance is reached. U.S. Pat. No. 6,132,429 describes using a radiofrequency device to weld blood vessels closed and monitoring the process by changes in tissue temperature or impedance. Nevertheless, these devices are generally unsuitable for the purpose of occluding a wound thereby enhancing long-term healing.

There has been an effort recently to identify biocompatible molecules which can be used as a “tissue solder”. Biomolecules such as fibrin, elastin, and albumin have been or are used to “glue” tissue to tissue. A number of patents describe the “activation” of these biomolecules to form “welds” through irradiation, often in the form of laser radiant energy, but sometimes in the form of ultrasound or radiofrequency waves. The applied energy is believed to denature the molecules, which then adhere to one another or cross-link upon renaturation thereby effecting a bond.

Over the past fifteen years, a significant amount of scientific research has focused on using laser heated “solder” for “welding” tissues such as blood vessels (1-2). Research has been done on laser tissue welding with albumin solders which is an improvement over conventional suture closure because it offers an immediate watertight tissue closure, decreased operative time, especially in microsurgical or laparoscopic applications, reduced trauma, and elimination of foreign body reaction to sutures, collagen-based plugs and clips. The procedure has been enhanced with the use of advanced solders, strengthening structures, concurrent cooling, and added growth factors, e.g., as disclosed in U.S. Pat. No. 6,221,068.

Use of lasers for tissue welding appeared very promising, however, the techniques have certain limitations. The laser energy must be manually directed by the surgeon, which leads to operator variability. Additionally, the radiant energy is not dispersed evenly through the tissue. The high energy at the focal point may result in local burns and the heating effect drops off rapidly at a small distance from the focal point. Finally, lasers are expensive and currently cannot be easily miniaturized.

U.S. Pat. No. 5,669,934 describes a method for joining or restructuring tissue consisting of providing a preformed film or sheet of a collagen and/or gelatin material which fuses to tissue upon the application of continuous inert gas beam radiofrequency energy. Similarly, U.S. Pat. No. 5,569,239 describes laying down a layer of energy reactive adhesive material along the incision and closing the incision by applying energy, either optical or radiofrequency energy, to the adhesive and surrounding tissue. Similarly U.S. Pat. Nos. 5,209,776 and 5,292,362 describe a tissue adhesive that is principally intended to be used in conjunction with laser radiant energy to weld severed tissues and/or prosthetic material together. U.S. Pat. No. 6,110,212 describes the use of elastin and elastin-based materials which are biocompatible and can be used to effect anastomoses and tissue structure sealing upon the application of laser radiant energy. The stated benefits, inter alia, are the biocompatible and ubiquitous nature of elastin.

U.S. Pat. No. 6,302,898 describes a device to deliver a sealant and energy to effect tissue closure. It also discloses pre-treating the tissue with energy in order to make the subsequently applied sealant adhere better. International Publication WO 99/65536 describes tissue repair by pre-treating the substantially solid biomolecular solder prior to use. U.S. Pat. No. 5,713,891 discloses the addition of bioactive compounds to the tissue solder in order to enhance the weld strength or to reduce post-procedure hemorrhage.

U.S. Pat. No. 6,221,068 teaches the importance of minimizing thermal damage to the tissue to be welded. The method employs pulsed laser irradiation and allowing the tissue to cool to nearly the initial temperature between each heating cycle. U.S. Pat. No. 6,323,037 describes the addition of an “energy converter” to the solder mixture such that optical energy will be efficiently and preferentially absorbed by the solder which subsequently will effect a tissue weld.

Induction heating is an industrial process often used to weld, harden or braze metal-containing parts in manufacturing where control over the heating process and minimized contact with the workpiece are critical. Inductive heating (3) is a non-contact process whereby electrical currents are induced in electrically conductive materials (susceptors) by a time-varying magnetic field. Basically, radiofrequency power is coupled to a conducting element, such as a coil of wire, which serves to set up a magnetic field of a particular magnitude and spatial extent. The induced currents or Eddy currents flow in the conductive materials in a layer referred to as the skin depth δ, given by:

δ=√(2ρ/μω),

where ω is frequency (rads/s), ρ is resistivity (ohm-m) and μ is the permeability (Webers/amp/m) which is the product of μₒ the permeability of free space and μᵣ the relative permeability of the material. For example, the skin depths at room temperature of copper is 0.066 mm and of 99.9% iron is 0.016 mm at 1 MHz electromagnetic radiation.

The consequence of current flowing is Joule heating. The skin-depth formula leads to the conclusion that, with increased frequency, the skin depth becomes smaller. Therefore, higher frequencies favor efficient and uniform heating of smaller components. In certain situations, localized heat can also be generated through hysteresis losses or frictional heating as the susceptor moves against physical resistance in the surrounding material. Consideration of Joule heating alone results in a formula for the power-density P (W/cm³) in the inductively-heated material:

P=4πH²μₒμᵣfM,

where H is the RMS magnetic field intensity (A/m), f is frequency (Hz), M is a power density transmission factor (unitless) which depends on the physical shape of the heated material and skin depth and diameter of the part to be heated (4-5).

M, which is equal to the product of F and d/6, where F is a transmission factor and d is the diameter of the part, can be shown to be maximally about 0.2 when the object diameter is 3.5 times the skin depth, and when certain other assumptions are made. Thus, for a given frequency, there is a diameter for which the power density is a maximum or, equivalently, there is a maximum frequency for heating a part of a certain diameter below which heating efficiency drops dramatically and above which little or no improvement of heating efficiency occurs. It also can be shown that the power density of inductively heated spheres is much higher than solid spheres of the same material.

Conventional applications of induction heating involve welding, hardening, brazing or forging metal components. Some applications have been reported which use the process to cure adhesives in bonding processes or for applying coatings. Recent development of adhesives composed in part of the susceptors used for welding or bonding conventional substrates is reported in U.S. Pat. No. 6,348,679. Devices and adhesive compositions used in bonding two or more conventional materials, typically metal substrates or films, through induction heating are disclosed.

There are only a few examples of the use of inductive heating in medical literature or for applications with biological materials. Principles of inductive heating have been applied to hyperthermia of cancer, whereby large metallic “seeds” are inductively heated using a coil external to the body (7). Additionally, a recent report described the use of induction heating to heat nanocrystals coupled to DNA in order to locally denature DNA for the purpose of hydridization (8). U.S. Patent Application Ser. No. 2002/0183829 describes inductively heating stents made of alloys with a high magnetic permeability and low curie temperature for the purpose of treating restenosing vessels. The effects of induction in tissue are not limited to tissue fusion. U.S. Pat. No. 6,573,491, and international publications WO 00/69515 and WO 00/77045 describe specific formulations, methods and devices where electromagnetic energy absorption is maximized relative to the surrounding medium, resulting in effects such as accelerated reaction rates and molecular mobility.

Common problems exist throughout the prior art. These include, for example, tissue damage due to uneven heating, unknown and/or uncontrollable thermal history, i.e., time-temperature profile, and relatively high cost. It is notable that a consistent means of treatment and control are desirable. The Code of Federal Regulations, 21 CFR 860.7(e)(1), establishes that there is “reasonable assurance that a device is effective when it can be determined, based upon valid scientific evidence, that in a significant portion of the target population, the use of the device will provide clinically significant results.” Devices that cannot be shown to provide consistent results between patients, or even within a patient upon multiple use, will have minimal utility and may not be approvable for broad use. Beyond devices, it is generally desirable to develop medical products with critical controls that can deliver precise results.

Tens of millions of venous access and puncture wounds are created each year as a result of catheterization procedures, biopsies, hemodialysis treatments and other procedures. Manual compression has been the standard of care for closure after percutaneous coronary interventions, but it requires prolonged bed rest, e.g. 4-12 hours, leading to delayed ambulation, significant medical staff time and associated higher costs. The routine administration of anticoagulant medication to prevent blood clots and stroke during the diagnostic or interventional procedure can further delay sealing the vessel and postpone ambulation. Complication rates as high as 12.5% for extraction atherectomy, and 11% for balloon angioplasty have been reported.

In recent years, several closure devices have been introduced to the market. Suture-mediated closure (SMC) devices push a shaft into the artery and use stitches to suture and close the puncture. When compared to manual compressison, the advantages of SMCs are a quicker time to hemostasis, 5 minutes vs. 25 minutes, and ambulation, 1 hour vs. 4-6 hours. However, these devices generally require a trained physician to insert the sutures, while most other closure devices can be managed by non-physicians. Reported complications include an increase in the number of access site infections, as well as pain and discomfort for the patient.

Some collagen-based closure devices use a biodegradable bovine collagen plug to form a coagulum at the access site. The two primary types are a plug, e.g. VasoSeal™ and a collagen plug with an anchor, such as Angio-Seal™. Hemostasis success rates range from 88%-100%, with an average success of 97%. When compared to manual compression, most studies show results similar to those for SMCs, i.e., a decrease in time to ambulation, 1 hour vs. 4-6 hours and time to hemostasis, 5 minutes vs. 25 minutes, and, furthermore, a 1 day reduction in hospital stay. Data on complications is mixed, with several studies showing minor complications comparable to compression, but an increase in major complications that require surgical repair. Other studies show an increase in minor complications. Collagen-based devices seal the vessel, but fail to seal the tract. In addition, manufacturers recommend that healthcare professionals not use the sealed vessel for a period of 3-6 weeks while the collagen plug is absorbed.

Manual pressure is the current standard of care for stopping post-dialysis bleeding as well. Limitations to manual pressure include: (1) the 10 to 20 minutes it typically takes to stop bleeding, occasionally taking up to an hour for difficult cases; (2) patients routinely receiving anticoagulant agents during their treatment thus lengthening the time required to stop the bleeding and leave the clinic; (3) applying too little pressure doesn't stop the bleeding, resulting in excess blood loss; (4) applying too much pressure causing the access to thrombose which requires additional interventions; and (5) manual pressure is labor intensive for the dialysis staff when patients are unable to hold their own site following needle removal. Success in rapidly and completely stopping the bleeding and sealing the tissue following the treatment can reduce complications such as infection and post-dialysis bleeding, as well as preserving the access.

Of the hundreds of thousands of Americans living with end stage renal disease, more than half undergo hemodialysis treatments 2-3 times each week. One challenge associated with successful hemodialysis is vascular access, the method used to access a patient's blood supply. Complications related to vascular access include thrombosis, stenosis, infection, pseudoanuerysm, limb ischemia and post-dialysis bleeding. The complications lead to loss of vascular access and the need for corrective surgery in the vast majority of patients twice per year. These corrective surgeries normally involve replacing an arteriovenous fistula or synthetic graft which provides access to the patient's blood supply.

The inventors recognize a need in the art for a tissue fusion closure devices that overcomes the deficiencies described supra and that could improve patient care and reduce costs, while supporting the expanded use of minimally invasive surgery. Such closure devices and methods of using the same work in a way somewhat similar to laser-tissue welding and maintain the clinical advantages of the approach, but eliminate the limitations. The prior art is deficient in devices and methods for minimally-invasive methods that use electromagnetic energy to controllably alter a biocompatible structure thereby making it adhere to tissue through molecular alterations and/or mechanical shrinkage. The present invention fulfills this longstanding need and desire in the art.

### SUMMARY OF THE INVENTION

The present invention provides methods, compositions and devices to inductively or conductively heat one or more substrates. Heat energy may induce an alteration in one or more of the substrates such as a conformational change.

The present invention is directed to a method for heating one or more substrates. Electromagnetic energy is applied to an antenna to generate an electromagnetic field. Heat energy from the electromagnetic field is delivered to the substrate(s) to heat them.

The present invention also is directed to a method of inducing an alteration in a substrate. Energy is delivered to an energy absorbing species to heat it. The energy absorbing species transduces the electromagnetic energy to heat the substrate thereby inducing the alteration therein.

The present invention is directed further to a device for the treatment of substrates. The device comprises a radiofrequency power supply, an energy absorbing species and a means for inductively applying the radiofrequency energy to the substrates.

The present invention is directed further still to a composition used in the treatment of one or more substrates. The composition comprises at least one reactant and at least one energy absorbing species.

The present invention is directed further still to a method of treatment for one or more substrates in an individual. The substrates are positioned and the composition described herein is applied to at least one of the substrates. Energy is applied to the composition whereupon the composition is cured thereby treating the substrates.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1A depicts the placement of exposed terminals attached to an electrical conducting element within a material which is altered upon the application of electromagnetic energy.

FIG. 1B is a cross-sectional schematic of a patch that is placed on the skin of an individual; the patch contains the electrical conducting element and a semi-permeable material.

FIG. 2 depicts the electrical conducting element with a linear geometry (FIG. 2A), with a coiled geometry (FIG. 2B) or consisting of small three-dimensional conducting nodes connected by fine linear elements (FIG. 2C).

FIG. 3A depicts a particular geometry of the electrical conducting element within a patch that is conducive to non-uniform heating.

FIG. 3B illustrates the theoretical temperature profile across the cross-section A-A of the patch in FIG. 3A.

FIG. 4A shows the conducting element positioned within a fusion composition in close proximity to the surface of the skin.

FIG. 4B shows the conducting element within a fusion composition in a coiled configuration to efficiently inductively absorb ambient radiofrequency energy produced by a coil attached to a radiofrequency power-source.

FIG. 4C depicts the conducting element within a fusion composition connected to a battery that is also incorporated into the patch.

FIG. 5 depicts a cross-sectional view of the patch showing that the fusion composition contains small conducting absorbers and an inductive coil around the fusion composition; the coil is powered by a battery regulated by an external switch.

FIG. 6 depicts a patch with an annulus for the weld connected to the terminals where a material or a medicament is contained within the annulus.

FIG. 7A depicts an arbitrarily shaped fusion composition containing an array of fine conducting elements. FIG. 7B depicts the placement of the array-containing fusion composition within the patch; a second part of the patch placed over the fusion composition contains conducting elements to heat the solder conductively or inductively.

FIG. 8 depicts the fusion composition containing an array of microneedles to alter skin surface prior to welding the fusion composition and the tissue. The fusion composition is surrounded by an annular electrode which incorporates an electrically conductive fluid.

FIG. 9A depicts the positioning of an active electrode within the fusion composition and the ground electrode emplaced on the stratum corneum distal to the fusion composition.

FIG. 9B depicts the positioning of both the active and ground electrodes within the fusion composition of FIG. 9A.

FIG. 10 illustrates the thermal history or temperature as a function of time of the fusion composition and contacting tissue. T1 is the ambient temperature of the fusion composition and contacting tissue, T2 is the threshold temperature T2 for the beneficial chemical change and T3 is the temperature at which irreversible thermal damage to extraneous tissue occurs. The duration of heating cycles illustrated may range from microseconds to many seconds.

FIG. 11 depicts a solenoid-type coil applicator carrying an electrical current and the resultant magnetic field lines.

FIG. 12 depicts a coil applicator that can be split thus allowing positioning of tissue in the interior of the coil.

FIGS. 13A-13C depict configurations of three flat pancake coils.

FIGS. 14A-14B depict a pancake coil with a non-planar geometry (FIG. 14A) an a conical spiral coil geometry (FIG. 14B).

FIG. 15 depicts a device that can be used to produce sealing of a catheter vascular access tract.

FIG. 16 depicts an applicator suitable for use in occluding cavities such as needle tracts.

FIG. 17 depicts an applicator suitable for use within hollow structures such as blood vessels.

FIGS. 18A-D depict an applicator which positions an anchor for use within hollow anatomical structures such as blood vessels.

FIGS. 19A-B depict different anchors for use within hollow anatomical structures.

FIG. 20 depicts an ovine blood vessel anastomosed with an activator, applicator and fusion composition.

FIG. 21 depicts a histologic section through a blood vessel anastomosed with the invention.

FIG. 22 depicts the increase in temperature of different fusion compositions as they are heated over a 60 sec interval.

### DETAILED DESCRIPTION OF THE INVENTION

One embodiment of the present invention provides a method for heating one or more substrates, comprising applying electromagnetic energy to an antenna to generate an electromagnetic field therefrom; and heating the substrate(s) via the electromagnetic field.

Further to this embodiment the method may comprise heating an energy absorbing species with a non-zero electrical conductivity via the electromagnetic field prior to delivering heat energy to the substrate(s) wherein the absorbing species deliver the heat energy to the substrate(s). The energy absorbing species may comprise matter which has a non-zero electrical conductivity. Such matter may be diamagnetic, paramagnetic, or ferromagnetic.

The energy absorbing species may be an an ionomer, a conducting polymer, an alkali metal, a transition metal, a lanthanide, or a metalloid or a combination thereof. In such instances the matter may comprise matter is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, or tungsten or alloys thereof. Furthermore, the matter may be matter is a metal nano- or micro-particle, a semiconducting nano- or micro-particle, a magnetic nano- or micro-particle, a polystyrene encapsulated metal particle, a buckminsterfullerene, or a liposome-encapsulated metal particle.

In aspects of this embodiment the electromagnetic energy is radiofrequency energy. The radiofrequency energy may have a frequency from about 20 kHz to about 40 GHz. The antenna may comprise at least one coil of electrical conductor. Examples of an electrical conductor are a solid wire or hollow tubing. Representative examples of an antenna are a single coil antenna, a double coil antenna or a solenoid antenna.

In all aspects of this embodiment the substrates may be a tissue, a cell, a biomolecule, a biologically active molecule, an adhesive, or a combination thereof. The biomolecule or biologically active molecule may be a protein, a lipid, a nucleic acid, or a carbohydrate. Additionally, the biomolecule or biologically active molecule may be a pharmaceutical, a biologic, a biomaterial, a diagnostic, or a biological marker. Further in all aspects heating the substrates may cleave a bond in at least one of said substrates, denatures at least one of said substrate or shrinks at least one of said substrates.

Another embodiment of the present invention provides a method of inducing an alteration in a substrate, comprising delivering energy to an energy absorbing species; heating the energy absorbing species via the energy; and transferring heat energy from the electromagnetic energy absorbing species to heat the substrate thereby inducing the alteration therein.

In all aspects of this embodiment the substrate may be inorganic or those substrates described supra. An example of an inorganic substrate is a shape-memory alloy. The energy absorbing species may be a susceptor or as the energy absorbing species described supra. The electromagnetic field may be generated via an antenna. Further in all aspects the radiofrequency energy, the antenna, and the alterations induced in the substrate are as described supra.

Yet another embodiment of the present invention provides a device for the treatment of substrates, comprising a radiofrequency power supply; an electromagnetic absorbing species; and a means for inductively applying the radiofrequency energy to the substrates.

In aspects of this embodiment the substrates may be biologicals, biologically active materials, tissues, or cells. The substrates may be in vitro. The power supply may generate radiofrequency energy as described supra. The means of inductively applying energy may comprise an antenna as described supra. The energy absorbing species are described supra.

Yet another embodiment of the present invention provides a composition used in the treatment of one or more substrates, comprising at least one reactant and at least one energy absorbing species. In aspects of this embodiment the reactants may be a protein, a lipid, a nucleic acid, or a carbohydrate. The reactants may comprise a pharmaceutical, a biologic, a biologically active molecule, a diagnostic, a biological marker or a combination thereof. The energy absorbing species are as described supra.

In a related embodiment there is provided a method of treatment for one or more substrates in an individual, comprising positioning the substrates; applying the composition described supra to at least one of the substrates; applying energy to the composition; and curing the composition thereby treating the substrate(s).

In aspects of this embodiment the substrate may be an implant, a bandage or those substrates described supra. Further in these aspects curing the composition may seal the substrates, may fill-in an opening in the substrates, may fuse the substrates, or may fix the substrates. Also curing the composition may denature at least one of the reactants or change molecular structure of at least one of the reactants. In all aspects the energy absorbing species, the radiofrequency energy and the antenna are as described supra.

As used herein, the term “weld” or, “fusion”, may be used interchangeably to represent bonding or attachment of one or more materials including sections of tissue to another section of tissue, to a fusion composition, to a dressing, or to a fastening device such as a clip, pin or staple.

As used herein, the term “curing” may be used to describe a process whereby applying energy to a substrate or substance changes the rheologic nature of the substrate or substance.

The present invention generally provides devices, methods and compositions for heating or inducing conformational changes in substrates placed in or found in organisms and cells. The substrates may themselves consist of biomolecules or bioactive molecules, tissues or individual cells, compositions applied to induce a certain effect and certain components of those compositions. Alternatively, the substrates may be molecules and structures that change conformation upon exposure to electromagnetic energy. The substrates may exist in a reaction vessel, in an organism or in tissue or cells present in or derived from an organism. The composition generally is a fusion composition substantially comprising a biomolecule or bioactive molecule, but additionally may comprise a non-biological material.

Biologically active molecules include biomolecules and otherwise biologically active molecules which can be naturally occurring in a living organism, or those which can have an influence on molecules in a living organism. Typically, such molecules may be found in or around cells and tissues, or may be supplied to living organisms, cells and tissues to achieve a desired effect or response.

Examples of biomolecules include proteins, carbohydrates or lipids found in cells or tissues. The biomolecules may be, although not limited to, structural, such as tissue structures composed of elastin or collagen or structural cellular components such as actin, myosin, or ribonucleoprotein particles, may be involved in catalysis, e.g. enzymes, or may be reactants, e.g., protease susceptible proteins, metabolized lipids. Examples of biological response modifiers are, antigens, protease inhibitors, enzymes, and metabolic inhibitors to name a few.

The device may comprise a source of radiofrequency (RF) energy coupled to an applicator which then produces an electromagnetic field, and the substrates, with or without the compositions, which interact with the electromagnetic field resulting in the production of heat or kinetic energy in the substrate and/or the composition. The device has three main components: activator, applicator, and optional fusion composition. Furthermore, the device may be incorporated within a patch.

The heating process may be used to heat substrates such as, but not limited to, tissue components or cells, molecular entities, e.g., proteins, lipids, carbohydrates, organic molecules such as pharmaceuticals, and inorganic materials, e.g. electro- and magnetically sensitive materials such as shape memory alloys. The heating process also may heat a liquid, solid or semi-solid fusion composition which may comprise the substrate or may be other than the substrate. The consequence of heat or other increased energy is molecular changes in the composition resulting in, for example, fusion with the adjacent tissue or changes in molecular conformations. The adjacent tissue may take part in the fusion process by also being altered by the transient presence of heat.

Applications include bonding or fusing, coagulating, molding, fixing, sealing and separating tissue components. For example applications may be fixing or curing a biological or bioactive material in place or may be fixing or curing substrates to adhere to structures. Additionally, applications may be used for bonding tissues or for filling defects in tissues. Sealing or welding a wound also is contemplated. A material or fusion composition that promotes the formation of a strong weld may be placed between layers of tissue, or between a tissue and a dressing that are to be welded. Heating the materials and/or tissues conductively or inductively effects a weld. A dressing or other fastener containing such material may be applied to the wound site and welded in place.

Activator

The power supply used may be a constant current or a constant voltage power supply or may be a modulated current or a modulated voltage power supply. The power-supply is able to produce radiofrequency energy with a power in the range 10^{-10,000} W and, depending on the application, may be more preferably in the range of about 100 to about 1000 W. The power-supply may typically operate at frequencies of 100 kHz to 40 GHz. Preferably the frequency range is about 100 kHz to about 5.8 GHz and, more preferably, the frequency range is between 350-800 kHz or at 869 MHz, 900 MHz, 2.4 GHz, 13.56 MHz or 5.8 GHz.

The best operating frequency depends, inter alia, on the nature of the fusion composition to be heated, the geometry and chemical composition of the material to be heated, tissue to be fused, or the cavity to be filled. Regulatory issues also may be a factor in the choice of frequency. The output impedance of the power-supply is preferably matched to the input impedance of the applicator, described below.

The power-supply has several safety features incorporated therein. For example, the output is optionally of low or moderate voltage, such as <240V, preferably no more than 50V, which is traditionally considered a safe voltage, however some applications require higher voltage. The device is shielded for emitted or received electromagnetic interference. Thermal switches are incorporated within the device to shut it down if overheating occurs. Fast breakers quickly cut off the output if a power-output transient occurs. Multiple interlocks are incorporated within the device which prevents running the device with the cover removed. A foot pedal optionally is incorporated in order to minimize the possibility of unintentional activation of the device.

Alternatively power may be supplied by technology related to commercially available electrosurgical generators and diathermy units. A continuously delivered magnetic field, such as is delivered through conventional induction heating and RF surgical devices may be used. Alternatively, a pulsed field may be provided as, for example, is generated by diathermy devices. Pulsed fields may alternatively be generated using capacitors in a cyclic manner to successively charge and release current to the respective RF generating devices. In this manner, large currents may be generated over brief amounts of time, with successive pulses. Pulsing the device in this manner also serves to minimize the effects of heat diffusion to surrounding tissue, over relatively long periods of time, by minimizing the duration of exposure to heating. Pulsing with a chaotic duty cycle can also reduce the time required to achieve the desired effect in the fusion composition.

Applicator

Applicator geometry greatly affects the distribution of the resultant electromagnetic field. There are several different possible designs for the applicator. The most efficacious design depends on the procedure for which it is used.

For conductive heating an electrically conductive element may be used. The electrically conductive element may terminate in exposed terminals and may be incorporated into a material. The conducting element may be coupled to a current source or high frequency voltage source through the terminals. The conducting element may be linear, coiled, or consist of small three-dimensional conducting nodes connected by fine linear elements. The conducting element is arranged within the material in a particular geometry to result in a non-uniform heat and, thus, weld across the area of the material. The conducting element may be or contain a metal, a protein, a ferromagnetic material, a pharmaceutical, a conducting polymer, or an ionic solution. Additionally, the conducting element may be embedded within a fusion composition or may be separate from but proximal to the fusion composition.

The electrical energy, i.e., a high frequency voltage or current, applied to the conducting element may be provided by at least one active terminal, a battery or an active electrode and a ground electrode. The active terminal may be an electrode array having a plurality of isolated electrode terminals. Both the active and ground electrodes may be embedded within a fusion composition. An active electrode may be embedded within a fusion composition and the ground electrode may be located distal to and external to the fusion composition. Optionally, the electrical energy may be modulated by a switch. Alternatively, the conducting element may have a geometry, e.g. a coiled configuration, that efficiently inductively absorbs ambient radiofrequency energy.

Additionally, a heating element with impedance greater than tissue may be used. The heating element is electrically positioned in series with a tissue, a conductive element and a second conductive element of lower resistance so that current flows through the tissue and the first element resulting in preferential heating of the element. A second conductive element with impedance less than tissue is in electrical series and grounds the current. Alternatively, a heating element with an impedance less than tissue is positioned electrically parallel with a tissue. Current flows through the tissue and heating element preferentially heating the element; a further conductive element with an impedance less than the tissue and the heating element taken together is in electrical series and grounds the current.

In the case of induction heating, a coil of wire, e.g., copper, can be connected to the activator in order to produce a strong and uniform magnetic field along the long-axis of the coil. The coil sets up an oscillating magnetic field, which inductively couples to a conductive material, i.e., conductive absorbers or susceptors, in the fusion composition or to the fusion composition itself. Heat is produced through physical movement of the conducting material and/or the establishment of eddy currents within the conducting material or the tissue and/or composition and/or hysteresis losses. The heat diffuses into the surrounding fusion composition and tissue thereby causing protein denaturation and subsequent molecular bonding thus effecting adhesion.

Such a coil is most suitable for inductively heating materials positioned within the turns of the coil. The coil can be made in such a way that it can be opened up thus allowing a tissue, such as a blood vessel, to be positioned within the coil which then closes and completes the circuit. Alternatively, the magnetic field can be externalized from the interior of the coil with the use of a core material, such as is used in transformers. The core material may be of a magnetic material or, optionally, a powdered magnetic material, so that heat production in the core is minimized. If required, the coil can be cooled by encapsulating it in a liquid-tight envelope, e.g., glass, through which a cooling fluid of low electrical permitivity, such as low viscosity mineral oil, can be circulated.

Other means of enhancing cooling may be achieved by using hollow tubing, such as copper, through which a cooling fluid, e.g., water, can be circulated. The advantage of such a scheme is that the dielectric property of the cooling fluid is irrelevent because it is contained within the conducting coils and not on the outside where it would be inductively coupled to the produced magnetic field. Optionally, the tubing material may be coated in a biocompatible non-stick material, such as teflon or a decomposible material such as pullulan, so that heated tissue will not adhere to the applicator.

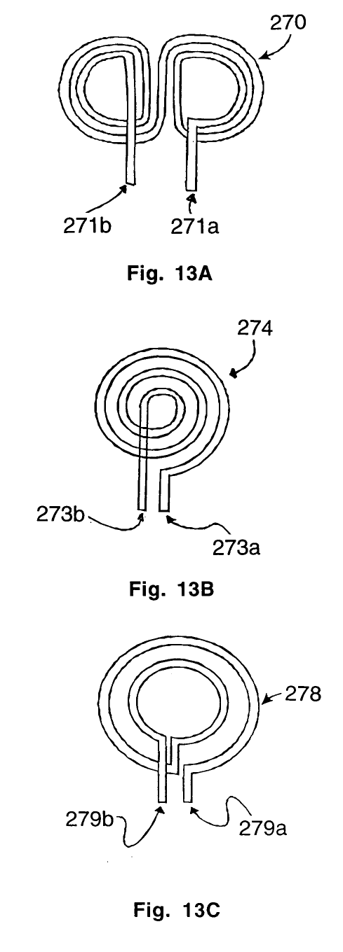

Other applicator designs allow for a relatively strong magnetic field to be produced exterior to the wire or tubing. For example, the designs shown in FIG. 13 are three examples of applicators whereupon the field is produced above or below the plane of the conductor. In FIG. 13A, the strongest field is produced below each separate coil while in FIGS. 13B and 13C, the strongest field is produced in a single position below the coil. Optionally, the applicator can be bent into a particular shape whereupon the distance between the material to be heated and the conductor that makes up the applicator is minimized. This provides for an efficient use of energy.

A ferromagnetic material, e.g. pole-piece, may be positioned partially in the magnetic field produced by the applicator, thereby allowing the field to be transferred to the end of the pole-piece thus producing concentration of the field lines and providing greater accessibility to the field. At high frequencies, it may be beneficial for this pole piece to be made substantially from powdered ferromagnetic materials or thin laminates in order to minimize undesirable heating in the pole piece itself.

Fusion Composition

The fusion composition may comprise formulations that may be used to secure tissues, cells, and other biological materials in place, or to one another. The formulations may act as, but not be limited to, an adhesive, a sealant or a filler. The fusion composition may be in a liquid, solid or semi-solid state and may comprise proteins and/or polymers dissolved or suspended in a biocompatible material such as water. The materials that make up the fusion composition are preferably biocompatible biological material, although the material may be non-biocompatible. The fusion compositions may be inductively or conductively heated and are able to produce a fusion in biomaterials.

The biocompatible proteins may be a protein, preferably e.g., elastin, albumin or collagen, and are typically present at concentrations of 0.1-100%, more preferably 50-75%. The fusion composition may be charged, by virtue of not being at its isoelectric point, or may have charged molecular species present which serve to interact with the electromagnetic field described supra. The fusion composition may serve as the substrate.

Generally, the fusion composition may comprise wholly or partially, for example, a biocompatible polymer, a protein such as albumin, elastin and/or collagen, or polysaccharides, e.g., cellulose, starch, chitosan, alginate, emulsan, pectin). Examples of biodegradable polymers are polylactide (PLA), polyglycolide (PGA), lactide-glycolide copolymers (PLG), polycaprolactone, lactide-caprolactone copolymers, polyhydroxybutyrate, polyalkylcyanoacrylates, polyanhydrides, and polyorthoesters. Examples of biocompatible polymers are acrylate polymers and copolymers, such as methyl methacrylate, methacrylic acid, hydroxyalkyl acrylates and methacrylates, ethylene glycol dimethacrylate, acrylamide, bisacrylamide or cellulose-based polymers, ethylene glycol polymers and copolymersm, oxyethylene and oxypropylene polymers, poly(vinyl alcohol), polyvinylacetate, polyvinylpyrrolidone and polyvinylpyridine. Optionally, protein primers, which are substances that exhibit groups that can cross-link upon the application of heat, can be added.

The protein may be a component of the fusion composition. Proteins are particularly attractive in tissue bonding applications in that they typically denature at temperatures less than 100° C. Denaturation can lead to cross-linking with other molecules, particularly other proteins, in the immediate environment while the proteins are either in the denatured state or upon their renaturation. Additional materials added to the composition formulations may result in greater flexibility, and tensile strength as well as optimum treatment times and temperatures.

The formulations will utilize commonly occurring tissue and proteins, such as albumin, collagen, elastin, but may also contain silk, lignin, dextran, or soyderivatives, poly-y-glutamic acid, combined with additives such as polyethylene glycol, glycerol, wax or hydrogel to improve the rheologic nature of the adhesive. Optionally, hyaluronic acid can be added to the composition to enhance the mechanical strength of adhesives, such as sometimes done in laser tissue welding, or pre-denaturation may take place before application of the composition at the treatment site.

Electively, other materials such as fibrinogen or chitin or chitosan may be added to the composition to provide hemostasis and/or some degree of immediate adhesion. Materials such as calcium phosphate or polymethylmethacrylate also may be used, most beneficially, when bony material is the tissue to be treated. Finally, pharmaceuticals such as an antiobiotic, may be beneficially added to the composition in order to provide some desirable pharmacologic event.

Optionally, destabilizing/stabilizing agents, e.g. alcohol, can be added as they have been shown to alter the denaturation temperature of the protein. For example, an increase in the concentraion of NaCl in a protein solution, which is referred to as “salting-in” proteins, can increase the denaturation temperature of certain globulins. An increase in the concentration of NaClO4 or “salting-out”, reduces the denaturation temperature (6).

When proteins are exposed to either liquid-air or liquid-liquid interfaces, denaturation can occur because the protein comes into contact with a hydrophobic environment. If allowed to remain at this interface for a period of time, proteins tend to unfold and to position hydrophobic groups in the hydrophobic layer while maintaining as much charge as possible in the aqueous layer. Thus, by ultrasonically adding bubbles to the composition will serve to lower the denaturation point of the mixture.

Conductive materials may be referred to as susceptors, which may also be substrates. The susceptor may additionally play a role as a transducer, whereby the energy transferred by induction is converted to heat or kinetic, e.g. vibrational, energy which results in a change in a particular target material. The susceptor material may be directly bound to the target or may be associated in the surrounding medium.

The conductive materials can be inductively or conductively heated and are added to the composition in amounts typically in concentrations of about 0.1 to about 25%. Higher concentrations may be used under circumstances where effects of the conductive materials on living systems are not a factor. The material may be composed of salts or other ionic substances or metals of variable size depending on the operational frequencies. The metallic material may be an alloy with a curie point in the range of 42-99° C. The metallic material is preferably biocompatible when working in a living system.

Generally, the range of useful particle sizes are from about nanometer size to macroscopic size particles up to about 1 mm wide. The particles may be, but not limited to, spheroidal, elongate or flakes. Alternatively, the conductive material may take of the form of a fine mesh or film, such as available from Alfa Aesar Inc (Ward Hill, Mass.).

Example of materials that may be useful by themselves, or in alloys, in the present method and composition are tantalum, niobium, zirconium, titanium, platinum, Phynox, which is an alloy of cobalt, chromium, iron, nickel, and molybdenum, palladium/cobalt alloy, magnetite, nitinol, nitinol-titanium alloy, titanium, which optionally may be alloyed with aluminum and vanadium at 6% Al and 4% V, tantalum, zirconium, aluminum oxide, nitinol (shape memory alloy), cobalt (optionally alloyed with chromium, molybdenum and nickel, or optionally 96% Co/28% Cr/6% Mo alloy), iron, nickel, gold, palladium, and stainless steel (optionally biocompatible type 316L).

The conductive materials may take the shape of a mesh, fibers, macroscopic and solid materials, flakes or powder. The conductive materials may be anodized and may further be encapsulated in materials such as liposomes, compounds such as calcium phosphate, polystyrene microspheres, pharmaceuticals, hydrogels, or teflon. The conductive materials may also be complexed with glass and ceramics. These complexes and encapsulating materials may minimize immune responses or toxic reactions to the conductor, could induct a desirable pharmacologic event, or could enhance the inductive coupling to the activating magnetic field.

The rheology of the fusion composition can be important. For example, producing the composition in a low-viscosity liquid form would allow injection through a cylindrical pathway such as a trocar or working-channel of an endoscope. A higher viscosity material can be applied to a tissue and will stay in place prior to activation. A solid formulation could be shaped, for example, as a tube, which then could be positioned in a tubular anatomical structure, e.g. blood vessel or ureter, thus providing mechanical support prior to activation.

Other shapes may be more appropriate for different procedures. For example, a flat-sheet of composition would be suitable for sealing a large area of skin or soft-tissue, while a solid cylinder could be most appropriate for placement in the cavity left behind after a cannula is extracted. The material alternatively may be molded into a tape, which can be applied to conform to the surface of planar and irregular-shaped objects. A pourous structure of the fusion formulation might be beneficial for the subsequent in growth of cells. It is contemplated that the conductive material itself, when distributed throughout the treatment area, would employ the endogenous proteins in production adhesion, thus precluding the use of an external protein in the formulation.

The composition optionally may have different additives depending on the material to which adhesion is required. For example, vascular graft materials composed of polytetrafluoethylene (PTFE) or Dacron may complex with denatured albumin. Alternatively, gelatinized PTFE, when used as one of the components of the fusion composition, could adhere to the PTFE in situ, thus effecting the desired result. Furthermore, heat-curable adhesives are included in the fusion composition. For example, heat-curable polymethylmethacrylate (PMMA) may be used to fuse bone components to one another, or to fill defects.

The fusion composition may incorporate a support lattice, such as can be made from, for example, porous calcium phosphate, polylactides, silk, PTFE or dacron, or a conductive material such as fine stainless steel mesh. The support material would allow for the fusion composition to be formed into a particular shape suitable for application to a particular anatomical structure. A conductive lattice would allow for inductive heating as well as mechanical support. Also, the efficiency of heating the fusion composition may be improved through the addition of ions in sufficient concentration to result in dielectric heating whereby ionic conductivity serves as a “bridge” between small particle conductive materials in the fusion composition.

The fusion composition may be in a formulation effective for membrane barrier function disruption. Specific formulations are chosen such that electromagnetic energy absorption is maximized relative to the surrounding medium. This may be accomplished through the addition of electromagnetic energy absorbers to the formulation. Further, many pharmaceutical or diagnostic compounds can be modified by either the addition of such energy absorbing groups or by selecting those that minimize absorption to maximize the effects of the electromagnetic energy on the formulation itself relative to the surrounding medium or tissue. Therefore, a new class of compounds is defined that have unique permeability, migration and deposition characteristics as a result of the addition of electromagnetic energy absorbing groups that function in the presence of, or following a treatment of electromagnetic energy as described herein.

These molecules possess different characteristics by virtue of the addition of groups or structures that absorb energy in a characteristic way. One result is that energy may impart momentum to the altered molecule causing it to move relative to the medium which contains it or applied energy may result in excitation of the molecule to cause a further change in that molecule. For example, rapid heating of a molecule, which preferentially absorbs energy relative to its environment, by radiofrequency energy, or by microwave energy, could result in direct activation of a specific activity or cleavage of a heat-sensitive linkage thereby releasing an active moiety.

The compounds and formulations are designed to include both physiologically active groups and molecular groups which maximize the absorbance or reflectance of energy to achieve the desired effect. This is analogous to prodrugs that release an active drug upon cleavage, usually enzymatic cleavage. Another analogy is found in photodynamic therapy whereby molecules absorb photons resulting in a transition from ground to an excited singlet state. This is followed by the transfer of energy to ground state oxygen in the nearby environment, whereupon the oxygen is excited to the singlet state, commonly known as ozone, which is toxic to cells.

Formulations may be chosen to effect deposition of a drug or a pool of drugs in a desired region of tissue or of cells. Modified molecules, such as pharmaceuticals with peptide or protein extensions, can be allowed to migrate to the region of interest, and may be activated to cross-link with the proteins in the target tissue. Alternatively, the complex may be allowed to be taken up by the cell, and then activated, preventing it from exiting the cell.

Pharmaceutically active compounds may be modified by the addition of groups that readily form a dipole or serve as energy “sinks” such that localized currents are induced when exposed to appropriate electromagnetic energy, such as radiofrequencies or microwaves. The addition of such groups would result in enhanced molecular vibration and/or migration of intramolecule electrons that may further weaken bonds in the modified molecule, or may result in a structural change to that molecule.

The carriers selected act as “sinks” for the energy whereby the energy is absorbed preferentially to the sink to limit exposure to the functional groups. Alternatively, molecules may be developed that have functional groups attached to a backbone molecule that is susceptible to cleavage when exposed to electromagnetic energy as described herein. Specifically, radiofrequency waves may result in excess vibration of groups as they absorb the energy. Using a linker that is susceptible to cleavage when its atoms vibrate in this way will result in the release of the functional group of interest which could be a pharmaceutically active substance. Also, magnetic fields alone may propel molecules through a medium or tissue based on intrinsic magnetic properties or by the addition of, inter alia, magnetic groups or metals which may be susceptors.

Patch

The device may be in a patch to be used externally or a small patch to be used endoscopically. Many different arrangements of the conducting elements, as described for the applicator, within the patch are possible and each arrangement would have a particular feature beneficial in certain circumstances. The conducting element may be arranged within the patch in a particular geometry to result in a uniform or non-uniform heat and, thus, weld across the area of the patch.

Electrical energy may be applied to the conducting elements within the patch via a battery incorporated into the patch. Given that the temperature rise necessary to cause the beneficial thermal alterations in the fusion composition are no more than about 60° C., and more likely only about 30° C., the energy available in the battery can be low enough that only a very small battery is required. This results in a convenient to use and yet disposable patch. A coil may be attached to a radiofrequency power-source external to and superimposed proximally to the patch will produce a magnetic field around the patch.

Upon being exposed to electromagnetic energy or to the heat generated thereby, the molecules in the material containing the electrically conductive element change in conformation, altering their interaction with each other or with molecules in the surrounding environment. For example, upon heating, protein may become more fluid, and flow into a second material, whereupon the molecules assume a different conformation upon cooling, thus enabling them to cross-link with molecules in the second material to form a weld or bond.

The second material may be composed of tissue, or may comprise, for example, a semi-permeable structure of carbon, of ceramic or of a polymer lattice such as a sol-gel or hydrogel. Additionally this second material may be an electrically conducting fluid or medicament that provides a pathway for electrical energy to reach the skin and effect tissue alteration, e.g., denaturation, thereby effecting a tissue-weld. Change in conformation upon exposure to an electromagnetic radiation is not limited to protein. Electo- and magneto-responsive materials, shape memory alloys and polymers are examples of other substrates that may be utilized in organisms in order to achieve a desired effect.

The patch may comprise the fusion composition. The fusion composition may be heated conductively or inductively via the conducting elements comprising the patch. The fusion composition itself may be the conducting element and is heated directly. For example, tissue fusion may be accomplished by applying metal particles to the interface between two tissue faces, or between tissue and another material, and, upon application of an alternating magnetic field, e.g., induction, the heat generated in the metal will diffuse to the surrounding tissues to create a weld. Alternatively, the fusion composition may comprise conductive absorbers or inductive transducers or susceptors, as described herein. Medicaments may also be incorporated within the fusion composition.

The conductive element is heated leading to thermal alterations of the fusion composition material which then effects a tissue-weld at the surface of the skin or of other tissue for endoscopic applications. The conducting element also may provide a means of measuring the heat generated in the system allowing for monitoring at a distal location. The conducting element may optionally be removed after the tissue fixation treatment, through physically withdrawing the element or through dissolving and absorption as a result of physiological processes. This may be accomplished, for example, through the use of conductive metals and polymers that are either solid or mixed in a semi-solid matrix.

The fusion composition may be heated by applying radiofrequency energy to a coil positioned around it or near it, thus causing a strong and alternating magnetic field within the fusion composition. For example, using a ferromagnetic material within the fusion composition, the fusion composition is heated by the external magnetic field until it reaches the Curie temperature of the ferromagnetic material. At this point the heating ceases until the material cools below its Curie temperature whereupon the heating cycle can be repeated.

It is additionally contemplated that the weld that holds the patch in place may take the form of an annulus. Positioned within the annulus is a material or medicament that is beneficial to wound healing. Examples of this material or medicament are a hydrogel or antibiotic ointment. Alternatively, the fusion composition may have an arbitrary shape and may or may not contain a medicament.

The fusion composition may incorporate an array of fine conducting elements such as, for example, metal or magnetic particles that may be heated by induction or a series of metal wires or mesh that may be heated conductively. The fusion composition may be cut with a scissors and placed over the wound to be treated. A second part of the patch is placed over the fusion composition and is used to inductively or to conductively heat the fusion composition through the application of radiofrequency energy via the terminals in the patch thereby effecting the tissue weld.

In order to effect a strong weld, it may be beneficial to pre-treat the skin surface before altering the fusion composition and tissue whereby the weld takes place. The patch may contain an array of microneedles within a fusion composition surrounded by an annular electrode which incorporates electrically conductive fluid. Upon the application of radiofrequency energy or a brief, e.g., a few microseconds, pulse or bipolar pulse of direct-current, tissue alterations take place in the skin concomitant with thermal changes to the fusion composition.

Additionally, electrodes incorporated within the patch can be excited by radiofrequency energy or a pulse or bipolar pulse of direct-current, whereupon a plasma is formed between the active and the ground electrodes. This creates alteration to the stratum corneum as well as beneficial changes to the fusion composition while leaving the epidermis unharmed. The plasma may also lead to the formation of transient cavitation bubbles that can also induce beneficial changes in the stratum corneum and/or fusion composition.

A safety interlock may be integrated into the patch such that the device cannot be utilized unless the interlock is engaged, and only under proper use. For example, the interlock could be mechanical, electrical or optical. In the “on” position or engaged, the device may be operational. In the “off” position or disengaged, the device would fail to be operational. This could prevent unauthorized use and would prevent the device from being used twice which would be unsanitary.

It is contemplated that inductive coupling most simply results in heating through the magnetization of particles or other ionic species, either with non-zero conductivity and magnetic permeability, and typically impregnated in a biocompatible fusion composition or adhesive. Alternatively, coupling may occur with particles in a tissue, or associated with biomolecules or bioactive molecules in a reaction vessel. Representatively, the composition may be composed largely of a protein, such as serum albumin, with the addition of a metal such as 300 mesh nickel flakes. The induced electrical currents produced in the particles results in heat which then conducts into the area immediately surrounding the metal, resulting in a “melting” of the adhesive and perhaps the adjacent tissue. When the adhesive cools, less than a second later, it forms a bond with the tissue, perhaps through cross-linking of the proteins.

It is contemplated that the adhesion effect is a consequence of the proteins in the fusion formulation bonding, perhaps by cross-linking, with other molecules in the protein formulation as they cool, as well as the proteins in the adhered tissue. This may be considered as a “bridge” between the molecules and a “scaffold” between the tissues. Furthermore, it is conceivable that the endogenous proteins in the tissue also were denatured and coagulated due to nearby heat production and that this may be critical to the adhesion strength.

In tissue, the temperatures needed to achieve protein denaturation, which may be a prerequisite for bond formation, range from about 45-85° C., and the heating times are very short since protein denaturation is essentially instantaneous once a critical temperature is achieved. Thus, the powers required for the present device and method are far less than those used in commercially available industrial induction-heating devices which are used for welding metals and plastics. Accordingly, the present invention can be produced for a fraction of the cost of commercial devices.

The present invention also provides a means to control the heating process by monitoring and regulating the heat generated or used in the system, so as to avoid overheating and damage to the materials, and to provide a uniform weld. The thermal history, i.e, temperature as a function of time, of the fusion composition and contacting tissue must be such that the beneficial chemical changes take place, e.g., denaturation, and yet little or no extraneous heat is produced which could otherwise lead to unwanted extraneous thermal damage. According to Arrhenius Rate Theory, the rate of a chemical reaction is exquisitely sensitive to temperature, but only linearly related to the time that a particular temperature is held. Thus, it is of benefit to quickly heat the tissue and fusion composition from their ambient temperature T1 to a temperature beyond the threshold temperature T2 for the beneficial chemical change, but not beyond the temperature T3 for irreversible thermal damage to extraneous tissue.

Once the critical temperature T2 is exceeded, the device quickly cools because of the small mass of the conductive heating elements or absorbers within the fusion composition whereupon the heating cycle can repeat. When the heating is done in a time more rapid than the time it takes the heat to conductively dissipate out of the heated tissue and fusion composition, then the total amount of energy used and heat produced during the process is minimized. Depending on the thermal properties of the heating elements and tissue, the duration of these heating cycles may be as short as microseconds or as long as milliseconds and the heating cycle can be repeated as many times as required to effect a suitable tissue fixation.

The tissue welding process also can be monitored by changes in the electrical properties of the electromagnetic circuit that is made up of the power supply, induction coil, material to be heated by the coil and the body. These changes may include but not be limited to changes in voltage or conductance or changes in the magnetic properties of a ferromagnetic metals and alloys, used as susceptors, in a fusion composition as it reaches its Curie temperature. Also the conductive or inductive heating process can be monitored by sampling changes in the first and/or second time derivative of the impedance of the tissue, comparing this derivative to zero and using this information to modulate the heating process.

Sequential rapid heating of biological targets, followed by cooling, has been shown to be a beneficial heating protocol in many laser therapies (1). This is because the temperature of the thermally sensitive target can be rapidly raised to beyond a threshold temperature, whereupon further heating is of no benefit. Instead the heat diffuses out into the surrounding tissue leading to undesirable thermal damage to uninvolved tissues. In order to maximize this benefit, it would be useful to rapidly pulse the fusion composition activator. Pulses, with length on the order of 1 ms or less would be beneficial, but pulses up to hundreds of milliseconds could also be useful. This can be accomplished with a bank of capacitors which, when charged, are discharged through the inductive applicator thus transiently producing a brief electromagnetic field pulse. Concurrently with the discharge of the capacitor bank is the charging of an independent capacitor bank, which can then be discharged while the first bank is recharging. In this way, a high duty-cycle can be achieved.

Other means of controlling the heating process are contemplated. The temperature of the treatment site is measured concurrently with treatment. Activation of a fusion composition substantially comprising, for example, bovine albumin requires a threshold temperature of about 75° C. to activate and fuse tissue. Monitoring of the temperature of the tissue during treatment can be done with an infrared thermometer, thermocouple or other thermoelectric transducer. The analog output of the thermometer can be digitized and sent to a controller, which then alters the output of the fusion composition activator in order to heat the composition to the critical threshold temperature, but not beyond where deleterious thermal damage may result.

Changes in particular physical properties, such as impedance, of the treated tissues may be directly monitored. When tissue is thermally altered, it's impedance changes. For example, alteration or removal of the stratum corneum of skin in an effort to enhance transdermal drug delivery also results in a dramatic reduction of skin electrical impedance. This reduction can be measured directly with electrical current and compared to a look-up table where the impedance is calibrated against temperature. The impedance can then be used to alter the output of the fusion composition activator.

Changes in the electrical load placed on the activation device as a consequence of changes in the electrical properties of the treated tissue may be monitored. The tissue being treated acts as part of the electrical load presented to the fusion composition activator. Changes in the electrical impedance of the tissue resulting from heating are sensed by the activator. When this phenomenon is calibrated against actual temperature measurements, this can be used to alter the output of the activator so that the critical temperature is reached.

A cooling system at the tip of the applicator or a cooling system allowing coolant to flow onto the target site may be added. For example, it may be necessary or desirable to heat tissue below a surface without heating the surface. One means is by spraying a coolant onto the surface simultaneously with the application. One example is heating cartilage lying beneath the skin surface.

Also described herein are methods and devices for inductively heating non-conventional substrates, i.e. biological materials such as cells, tissues and molecular entities, in order to cause conformational changes that result in unique properties with regard to tissues. In particular, the principles of induction heating are applied to treat biological materials and cause them to join to one another or to non-biological materials. Alternatively, induction heating may be used to separate tissues. These methods may be used to anastomose tubular structures such as blood vessels or ureters.

The inductive heating methods use devices providing radiofrequency energy to generate an electromagnetic field to produce heat substantially within a fusion composition. The fusion composition may function as a fusing or bonding agent between two or more elements of a tissue or as a sealing agent to seal a sinus within a tissue, such as a vascular access defect or other defects within a tissue. For example In these methods the fusion composition may comprise a conducting absorber or susceptor to transduce the electromagnetic field to heat production within the fusion composition. The methods encompass a means of monitoring the amplitude or persistence time of the electromagnetic field generated during application of the method.

Additionally, the fusion composition may function to effect a weld between a tissue and at least one other substrate, including the fusion composition. The method provides a means of monitoring the extent of the weld, such as via feedback monitoring of temperature or impedance. The substrate optionally may comprise, but not be limited to, a tissue or a material commonly used in medical implants.

More specifically, one method provides a means of cauterization and dissection of a tissue without contacting the tissue. A conductive composition is applied to the surface of a substrate, such as a tissue which is leaking fluids, e.g. bleeding. The composition is heated through induction using the devices described herein to a point where the tissue beneath the composition is cauterized as a result of the heat generation. Application of additional heat can be used to cause separation of the tissue, with simultaneous cauterization.

Similarly, a conductive composition is applied to the surface of a substrate, for example, a tissue to be dissected. The composition is heated through induction to a point where the tissue beneath the composition is separated as a result of the heat generation. As a result of heating and separation, the tissue beneath the composition is cauterized, thus limiting bleeding.

The methods and devices described herein may be utilized to induce conformational changes in biomolecules and bioactive molecules such that they may react in a unique manner or such that the rate of reaction is accelerated. The enhanced reactions are useful in a range of biological applications, including, but not limited to, wound healing and tissue fusion, deposition of pharmaceutical agents, fixation of implants and tissues, development of multi-laminate and multi-vesicular delivery agents, and cosmetic alteration of tissues.

In one method a device utilizies a radiofrequency-generated electromagnetic field to inductively transfer energy to reactants thus accelerating a biochemical reaction. One or more of the reactants taking part in the biochemical reaction may have a molecular or macroscopic absorbing species or transducer, i.e., susceptor, linked to it, or in close proximity to it, for the purpose of enhancing the transduction of energy from the electromagnetic field to the reactants. Optionally, the reactants may be proteins, the molecular transducer or susceptor may be an ionomer and the macroscopic transducer may be a metallic nanocrystal or particle.

In a similar method the device is utilized for inductively transferring energy to reactants, in vivo or in vitro, thus accelerating a biochemical reaction. This reaction has multifold beneficial uses. For example, the reaction results in the fusion of molecular species to one another, in the modification of an artificial or naturally occurring membrane to increase permeability thereof, or the release of an active drug moiety from a pro-drug.

For example, it is known that the efficacy of chemotherapeutic drugs in inducing lethal damage to malignant cells increases with the increasing time that the drug is present adjacent to or within the cells. An ongoing problem in cancer therapy is getting malignant cells to retain chemotherapeutic drugs. A novel and potentially powerful form of cancer therapy would involve the in situ inductive biomolecular alteration or activation of a chemotherapeutic drug/magnetic particle conjugate which would serve to make the cancerous tissue retain the drug. Migration of the altered drug from the desired site of action would be minimized. It is contemplated that the decreased migration is the result of direct binding of the altered molecule to another species or through an alteration of its mobility characteristics.

Additionally, the methods and devices are utilized as a means of inducing conformational changes related to curing, denaturing or other alterations of biological material either in molecular or tissue form. Examples include the alteration of molecules, such as, denaturation of proteins and the release of active drug or biologic agents from pro-drugs or pro-biologics with heat-cleavable or molecular vibration sensitive linkages. Examples also include the enhanced binding ability of protein and other molecules, including certain molecules in receptor-ligand interactions.

Similarly, the methods and devices are utilized to aggregate proteins and other biological materials to form structures which are useful, for example, in rebuilding tissues, fusing and fixing tissues and for creating aggregates of liposomes and proteins for drug delivery and deposition. For example, spherical or planar protein or lipid composites, including liposomes, may be fused together using inductive heating to form multi-laminate or multivesicular materials. Multi-vesicular liposomes may be produced in this way. Such multi-vesicular liposomes and multi-laminate materials are useful for as, inter alia, drug delivery agents, or carriers.

The protein-susceptor combination may be used to connect membrane structures such as those in liposomes or in living cells. For liposomes, modifications are made to the formulation to include protein and susceptor in the liposomal membrane. Upon activation, the proteins in the membranes cross-link to form aggregates of one another and of the liposomes. By controlling time and energy applied, different sizes of these multi-vesicular liposomes may be formed. Such multi-vesicular liposomes are useful in deposition drug delivery as they deposit in a region and are slow to dissolve or resorb, thus resulting in slow, sustained release of the contents. Clinical applications of either single or multi-vesicular liposomes also include filling the carrier with a pharmaceutical substance, allowing it to localize in a particular region of tissue, then activating the modified liposome resulting in crosslinking of the membrane proteins to proteins present in the membranes of tissue cells of the region. Thus, the liposomes became deposited on the tissue of choice.

Furthermore, these methods and devices may be utilized to form deposition drug delivery and measured release agents comprising multi-laminate sheets or multi-vesicular liposomes. The sheets may be incorporated with drugs and the structure may be deposited in a specific tissue region or cavity. Degradation of the vesicles or laminates over time may result in the release of drugs until dissolution is complete. By varying the number of laminates or vesicles, the duration of degradation may be controlled.

Similarly the methods and devices described may be utilized to fuse biomolecules, bioactive molecules, laminates and multi-vesicular liposomes to tissues. An example of an application of this embodiment includes the deposition of biomolecules and carriers to tissue where the biomolecule or carrier may comprise a protein that is capable of cross-linking to surrounding tissue or cellular proteins. In this example, the biomolecule may bind intracellularly or extracellularly. The biomolecule or carrier may thus be localized in a desired region, such as a tissue or in a cavity. This application, for example, provides a means for localizing chemotherapeutic agents at the site of a tumor or intracellularly in certain situations, thus increasing the likelihood that a target may receive therapeutic benefit.

Furthermore, the methods and devices provide for the formation of certain structures that are manufactured using inductive processes. These structures may also take benefit from induction during their use in biological environments. The structures may include, but are not limited to, a scaffold, or porous and filamentous structures comprising biological materials such as proteins. Additional components, such as energy absorbing species, e.g. susceptors, may be included in the structure and may act as transducers. The susceptor material further may be particulate or of a desirable shape, such as, for example, a coil or rod.

These structures are useful, for example, as support during the reconstruction of tissue, filling in or sealing tissue defects, or in fixation of tissue with tissue or implants. These structures may allow for growth of the tissue in and around the support. For example, during bone reconstruction, it may be desirous to fill defects using a scaffold or porous matrix comprising protein and calcium carbonate such that the matrix provides initial integrity and strength, but also allows for tissue to grow into pores over longer periods of time.

The invention further provides irregular structures that may preferentially interact to varying degrees with the magnetic field produced by the applicator. Thus gradients of susceptor material may be positioned in a structure such that the material may be heated at different rates when exposed to a constantly changing field or may heat relatively linearly when exposed to a changing field. For example, a linear filament may comprise a protein and susceptor where the susceptor is at a greater concentration proximally than distally, with regard to the applicator. Thus, an increased concentration of susceptor distally from the applicator results in more efficient heating, compared to proximal sections that are at low susceptor concentration. This, in effect, compensates for the decreasing field at distances from the applicator.

The applications described herein are not limited to cells and tissues of animals. The methods, devices and formulations described herein have application in other living systems, or those derived from living systems. For example, plant tissues may be grafted to one another using the techniques described in this invention. Such grafting may be used to hybridize plant species. Cells of various organisms may also be fused to one another to create multi-cellular constructs, or for fusion of cellular components.

It is contemplated further that the methods, devices and fusion compositions described herein may have medical applications. Examples are, but not limited to, fixation, tissue reconstruction and/or other aesthetic procedures.

Fixation of bones to bones, or bone-implants to bone, can be a very problematic procedure in surgery. For example, fixation of hip implants into femurs necessitates the use of cyanoacrylate glues, which bond nearly instantaneously upon contact. Incorrect positioning of the implant at the time of glue setting results in compromised results and may even require prosthesis removal, which itself may result in fracturing of the femur. The same problem can be said of bone-to-bone fixation. The fusion compositions of the instant invention are a significant improvement over the existing technology in that the bone or implant to be fixed in place can be coated in the fusion composition, or the site of fixation can be coated in the fusion composition, and correct positioning can be confirmed prior to activation of the composition with the electromagnetic field. This minimizes the chance of incorrect positioning.

Incorrect positioning prior to fixation can be a problem in itself in fixing soft tissues or therapeutic materials. For example, in treating incontinence, the bladder and urethra are stabilized by a surgery performed by placing sutures between the ligaments and tendons that support the pelvic organs and then tying them to the pubic bone. Alternatively, tissue may be sutured in place beneath the urethra and bladder neck for support. This can prevent accidental release of urine from the bladder when laughing, sneezing, or coughing. The methods of inductive heating to fix, bond, weld, or fuse tissue used in these procedures would fix the tissues in place.

Skin and other graft fixation, as well as securing bandages, is also an important potential use of the invention. Fixing grafts and bandages in place with the instant invention has the added benefit of minimizing the movement associated with securing tissues or bandages in place that have different flexion properties than the surrounding tissues. For example, bandages positioned over pressure-sores or the cutaneous ulcerations that can result from diabetes often move and rub the wound, thus preventing healing and perhaps even accelerating further decomposition of the wound.

The use of alloplastic implantable materials, often consisting of a polymeric shell filled with saline or silicone, are very commonly used in soft tissues, but can give rise to significant problems in surgery. They are commonly used to repair traumatic wounds, congenital deformities, and cosmetically unappealing appearance. However, an implant can occasionally become malpositioned thus requiring a revision surgery. Sutures are sometimes used to fix the implant in place, although migration of the implant, which can lead to poor cosmesis or therapeutic effect, can still happen. Autogenous fat transplants have been used for decades but have largely been replace by, injectable bovine collagen, first available in 1981. Zyderm and Zyplast (Collagen Corp, Palo Alto, Calif.) are now available and have been used with excellent safety records, with the major drawback of rapid absorption as illustrated by no histological evidence of Zyplast by 6 months and Zyderm by 3 months. Again, fixation of the implant in place, with or without the use of fusion composition, would both minimize post-implant malpositioning and undesirable rapid decomposition and absorption.

Reconstruction of tissue is done for both cosmetic, as well as therapeutic reasons. For example, chondral transplantation is sometimes done when the knee cartilage focally degenerates, perhaps due to local acute trauma. It is possible to take plugs from the interior aspect of the damaged knee and then position them close together in the defect thereby allowing reformation of the collagen surface. In other reconstruction situations, it is sometimes possible to simply position viable collagen tissue at the defect in the hope that tissue regeneration will take place, thus resolving the lesion. In either case, migration of the transplant must be minimized.

Plastic surgeons sometimes use porous hydroxyapatite implants which when implanted into the body allow normal tissue integration to occur. Also a non-porous paste comprising hydroxyapatite may be used. The instant invention can be used to fix either the plugs, hydroxyapatite or collageneous tissue in place. Here, the fusion composition, optionally with hydroxyapatite added to enhance oseointegration, is applied first to the plugs, or mixed in with the collagenous tissue, and is positioned manually to take advantage of the fact that the fusion composition can be made highly viscous and so will stay in place prior to activation. Once correct positioning is confirmed, activation of the fusion composition occurs, either with an endoscopically positioned coil applicator or transcutaneously with an external coil applicator. Activation of the fusion composition eliminates migration of the transplant.

Thermal shrinkage is a relatively new procedure used to treat small tears of the anterior cruciate ligament (ACL), i.e., instability in the knee. The collagenous tissue in the ligament is “shrunk” in size using electrosurgical units, or holmium-YAG lasers. This shrinking results in tightening of the ligament, thus minimizing instability, and minification of the extent of the tear. Using the present invention, the ACL is heated transcutaneously or minimially-invasively with a small activation coil. The endogenous charged species may interact with the activation field, thus producing heat. Alternatively, the fusion composition could be applied to the ACL exactly where the heat and shrinkage is desired, thus enhancing the selectivity of the treatment and avoiding the heating of uninvolved tissues.

Keratoplasty is a procedure whereby the cornea is reshaped with a laser, or experimentally with heat from a radiofrequency device, thus changing the cornea's refractive characteristics and so, for example, reducing hyperopia. The instant invention provides a new way to perform this procedure. For example, by utilizing endogenous charged species in the cornea, an ambient electromagnetic field could be applied in a non-contact fashion, leading to heating of the charged species, and subsequent shrinkage of the corneal tissue. With an electromagnetic applicator of particular shapes, non-uniform electromagnetic fields can be created thus inducing non-uniform heating patterns and tissue shrinkage.

There are multifold aesthetic procedures which could be accomplished by the instant inventions. For example, hair removal, wrinkle removal, scar revision, facial resurfacing, port-wine-stain therapy, collagen reshaping, and tattoo removal are aesthetic procedures. Currently in some of these procedures, chemicals are used to elicit the beneficial response. Alternatively, lasers or electrosurgical devices can be used to heat the tissue, e.g. skin, thereby causing shrinking, coagulation and a cascade of healing events leading to the desired response. There is a new technique that involves a radiofrequency plasma and purportedly does not produce heat (Visage, ArthroCare Corp., CA). Chemicals are relatively difficult to use, lasers are very expensive, and the electrosurgical devices, e.g. Thermacool system (Thermage Inc., CA) is a contact system requiring the patient to wear a grounding pad and so has the same risks seen in classical electrosurgery.

The present invention may be used in tissue treatment procedures with the added benefits of being non-contact, inexpensive, and with only minimal operator skill necessary. It is contemplated that the radiofrequency energy generated through induction may result in hysteresis in molecular entities within the tissues themselves. This effect is accentuated in the presence of ions or when high frequencies are applied and results in localized heating in the regions surrounding the affected molecules.

The hair removal technique of choice involves the use of a laser in selectively targeting melanin. Each hair has 3 distinct components of which one is the bulb, which lies near the insertion of the erector pili about 4 mm beneath the surface of the skin, where pluripotential cells cause growth of the hair follicle and where melanocytes also are present. During anagen, which is the active growth phase in the hair growth cycle, at which time hair matrix cells divide rapidly and migrate outward from the shaft and the melanin load is at its highest, laser therapy is effective. Basically, the laser radiant energy is converted into heat in the melanin thus causing irreparable thermal damage.

The problems are that the hair must be darker than the surrounding skin, hypopigmentation or hyperpigmentation may result, and the radiant energy of the laser must penetrate to at least 4 mm. The present invention, when used for hair removal, has the added benefits of much deeper penetration of the electromagnetic energy, and works independently of melanin content and skin color. With the use of a fusion composition, either injected below the surface of the skin or spread on the surface of the skin where hair removal is desired, allows for selective thermal damage.

There are other cosmetically unappealing situations that would benefit from the use of the instant invention. Skin resurfacing, for wrinkles, acne, scar revision, inter alia, typically involves chemicals, pneumatic crystals or laser radiant energy, which ablate the top layers of skin. This removes hypertrophic and hypotrophic structures, causes collagen melting and induces a cascade of healing events and new tissue generation that eventually lead to the desirable cosmetic endpoint. In laser-assisted tattoo removal, darkening of the skin often occurs, which is a consequence of the laser-induced conversion of ferric oxide to ferrous oxide in the tattoo ink resulting in an insoluble black pigmentation within the skin. Furthermore, allergic reactions sometimes occur with laser treatment of tattoos purportedly caused by altered antigenicity of the tattoo pigment by the laser light energy.

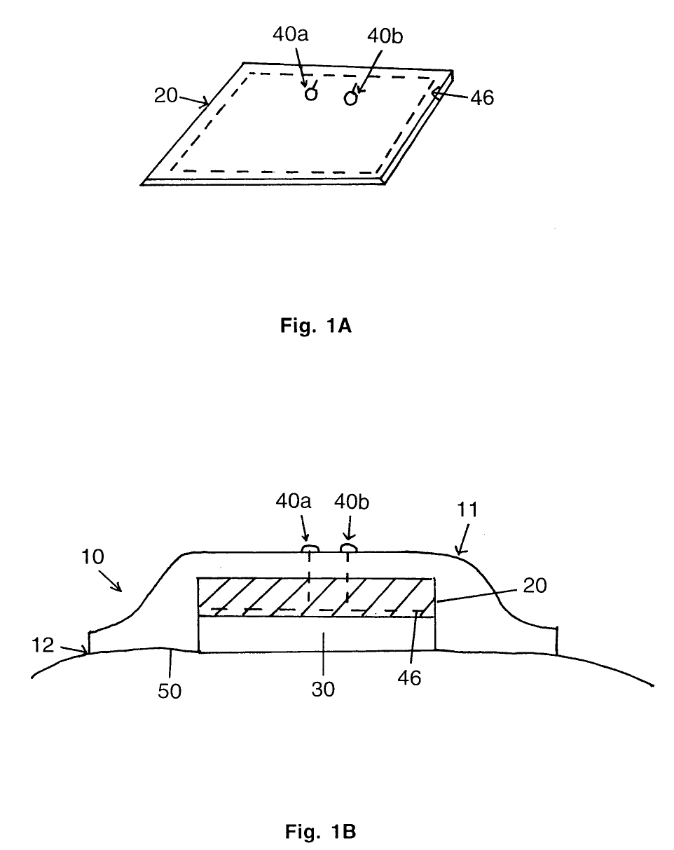

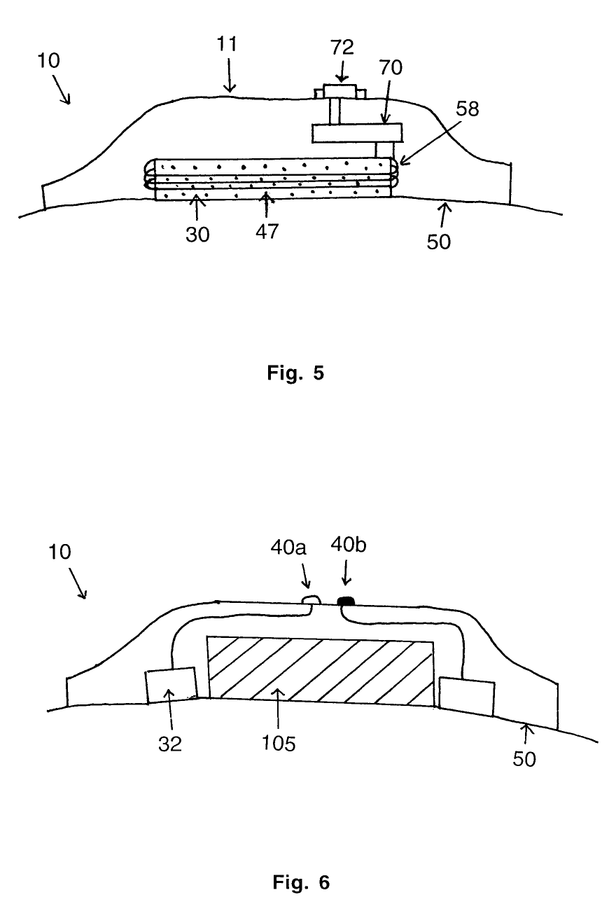

As described below, the invention provides a number of therapeutic advantages and uses, however such advantages and uses are not limited by such description. Embodiments of the present invention are better illustrated with reference to the FIGS. 1-21, however, such reference is not meant to limit the present invention in any fashion. The embodiments and variations described in detail herein are to be interpreted by the appended claims and equivalents thereof.

FIG. 1A shows a material 20 which may be a semi-solid matrix incorporating a conducting element 46. The conducting element terminates at exposed terminals 40a,b. The terminals 40a,b may couple the conducting element 46 to a current source or high frequency voltage source (not shown).

In FIG. 1B the material 20 containing the conducting element 46 is incorporated into a patch 10. The patch 10 has an upper surface 11 on which the terminals 40a,b are located and a lower surface 12 which contacts the surface of the skin 50. The patch may optionally have an adhesive (not shown) for temporary adherence to the tissue. The material 20 containing the conducting element 46 is contained within the patch 10 and placed in contact with a fusion composition 30 within the patch 10 which is in contact with the skin 50 such that the fusion composition 30 is sandwiched between the material 20 and the skin 50.

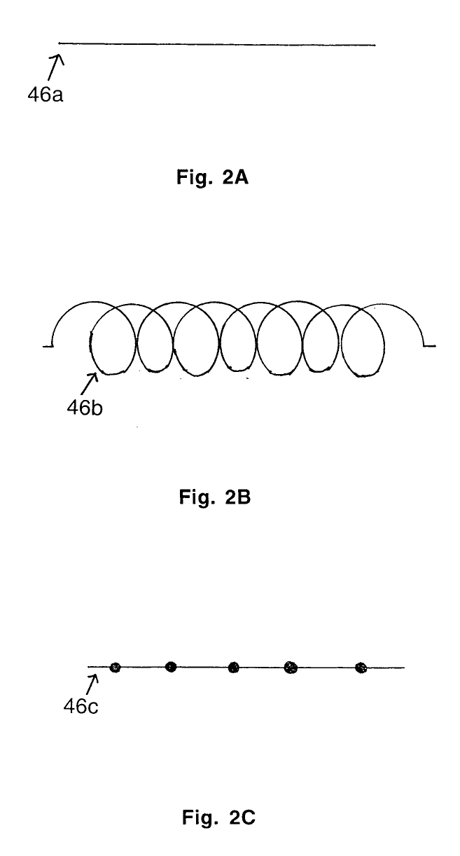

With reference to FIGS. 1A and 1B, FIGS. 2A, 2B and 2C depict possible geometries of the conducting element 46. The conducting element 46 may be linear 46a, coiled 46b or consist of small conducting nodes which are connected by fine linear elements 46c. It is to be noted that reference to conducting element 46 includes, but is not limited to, geometries 46a, 46b and 46c of the conducting element 46 unless specifically indicated otherwise.

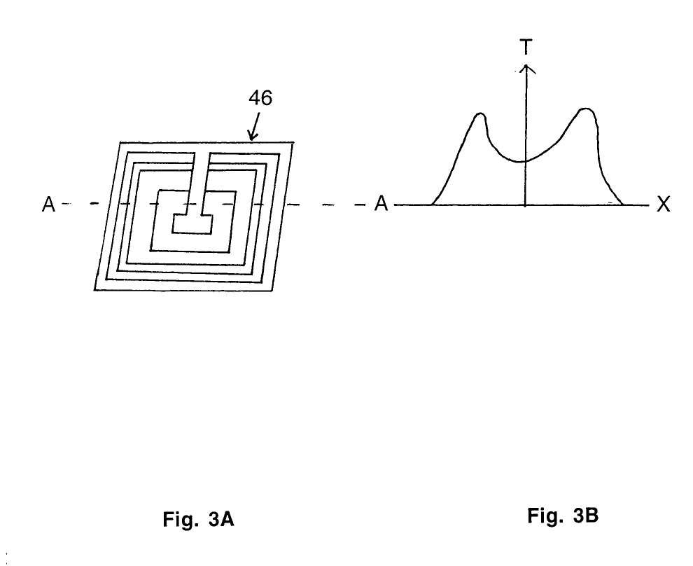

FIG. 3A depicts an arrangement of the conducting element 46 in a particular geometry that results in a non-uniform heating and, thereby, weld across the area of the conducting element 46. FIG. 3B illustrates a theoretical temperature profile across a cross-section A-A of the patch 10 showing the non-uniformity of the temperature.

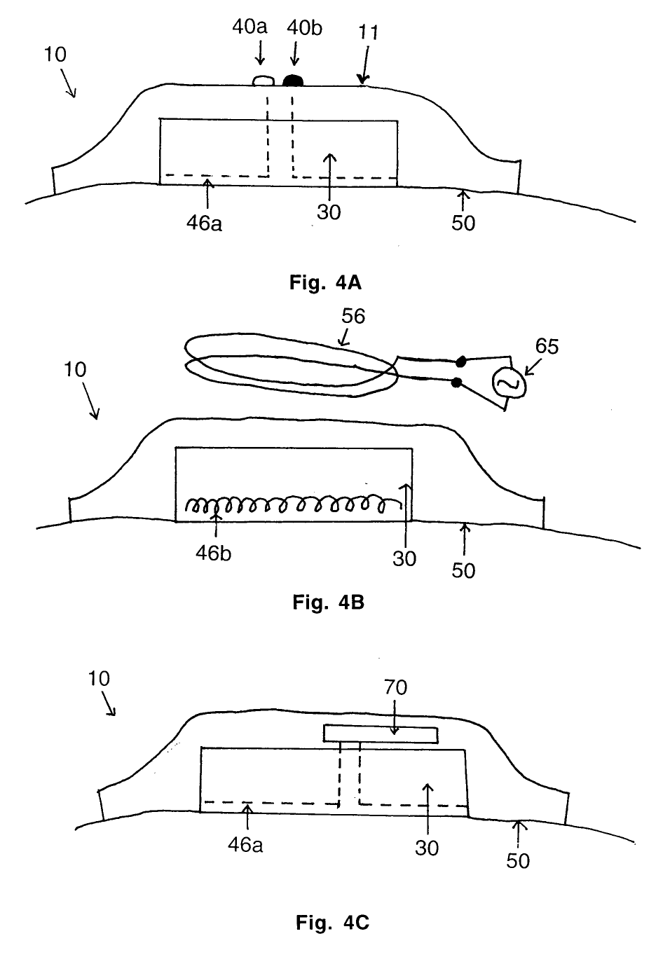

Still with reference to FIG. 1B, FIGS. 4A-4C depict a patch 10 having the conducting element 46 within the fusion composition 30 with various means of conductively or inductively heating the conducting element 46. In FIG. 4A a patch 10 comprises a fusion composition 30 placed within the patch 10 such that the patch 10 and the fusion composition 30 are in contact with the skin 50. The conducting element 46a is positioned within the fusion composition 30 to be in close proximity to the surface of the skin 50. The conducting element 46a terminates at exposed terminals 40a,b located on the outer surface 11 of the patch 10. The terminals 40a,b may be coupled to a current source or high frequency voltage source (not shown) as in FIG. 1B.

In FIG. 4B the fusion composition 30 contains conducting element 46b located proximally to the surface of the skin 50. The conducting element 46b inductively absorbs ambient radiofrequency energy generated by a coil 56. The coil 56 is external to the patch 10 and superimposed proximally to the upper surface 11 of the patch 10. The coil is attached to a radiofrequency power source 65.

FIG. 4C depicts a patch 10 with fusion composition 30 having a conducting element 46a as in FIG. 4A. The conducting element 46a terminates in a battery 70 incorporated into the patch 10 but external to and superimposed proximally to the fusion composition 30.

With continued reference to FIGS. 1B and 4C, FIG. 5 depicts a patch 10 comprising a fusion composition 30, placed proximate to the surface of the skin as in FIG. 4C, containing small conducting absorbing elements 47. The absorbing elements 47 are inductively heated by radiofrequency energy supplied to a coil 58 emplaced around the fusion composition 30. The battery 70 powers circuitry (not shown) that delivers the radiofrequency energy to the coil 58 and is modulated via a switch 72 connected to the battery 70. The switch 72 is located on the upper surface 11 of the patch 10.

FIG. 6 depicts a patch 10 comprising an annulus 32 in contact with the surface of the skin 50 and which is connected to terminals 40a,b. Emplaced within the area circumscribed by the annulus 32 is a material or medicament 105 in contact with the surface of the skin 50.

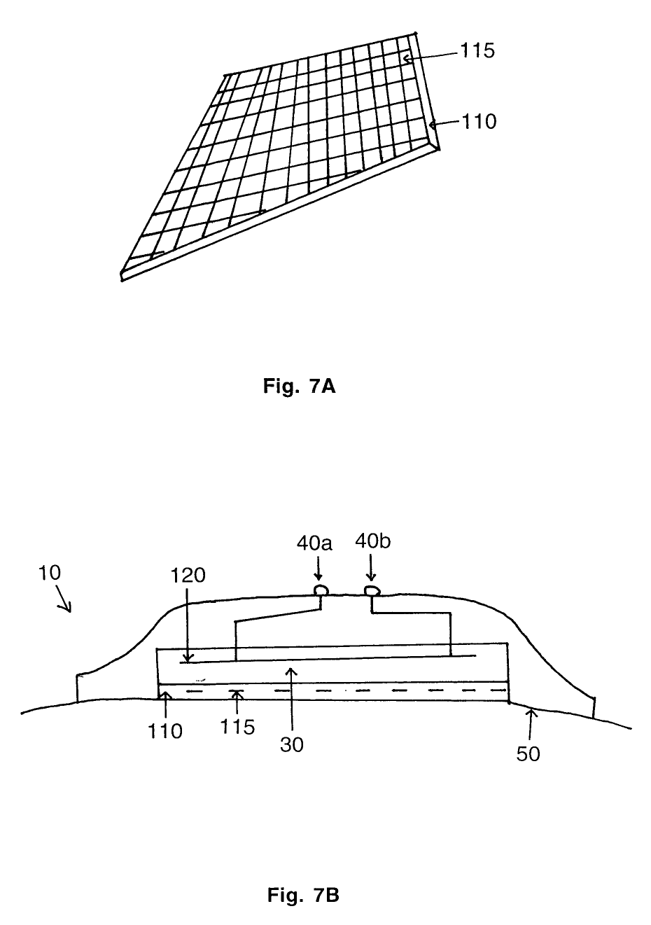

FIG. 7A depicts a fusion composition 110 having an arbitrary shape and capable of being cut with scissors or other sharp instrument. The fusion composition 110 incorporates an array of fine conducting/heat absorbing elements 115. As shown in FIG. 7B, the fusion composition 110, cut in a desired shape, is contained within the patch 10 and placed over a wound on the surface of the skin 50. Material 30 which may be composed of a semi-solid matrix connected to exposed terminals 40a,b at element 120 is placed over the fusion composition 110 and 120 is connected to exposed terminals 40a,b. The element 120 either conductively or inductively heats the fusion composition 110 via application of radiofrequency energy to terminals 40a,b which thus effects a weld at the skin 50.

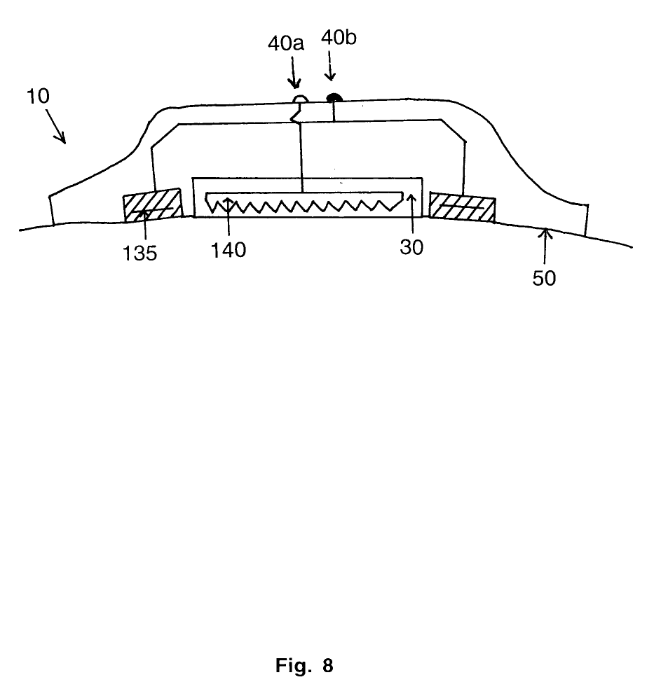

FIG. 8 depicts a patch 10 containing a fusion composition 30 placed on the skin 50. The fusion composition 30 contains an array of microneedles 140 proximate to the skin 50 which are connected to terminals 40a,b. An annular electrode 135 incorporating an electrically conductive fluid (not shown) also is connected to terminals 40a,b. Radiofrequency energy or a brief pulse or bipolar pulse of direct current through terminals 40a,b results in both tissue alterations of the skin 50 and thermal changes to the fusion composition 30.

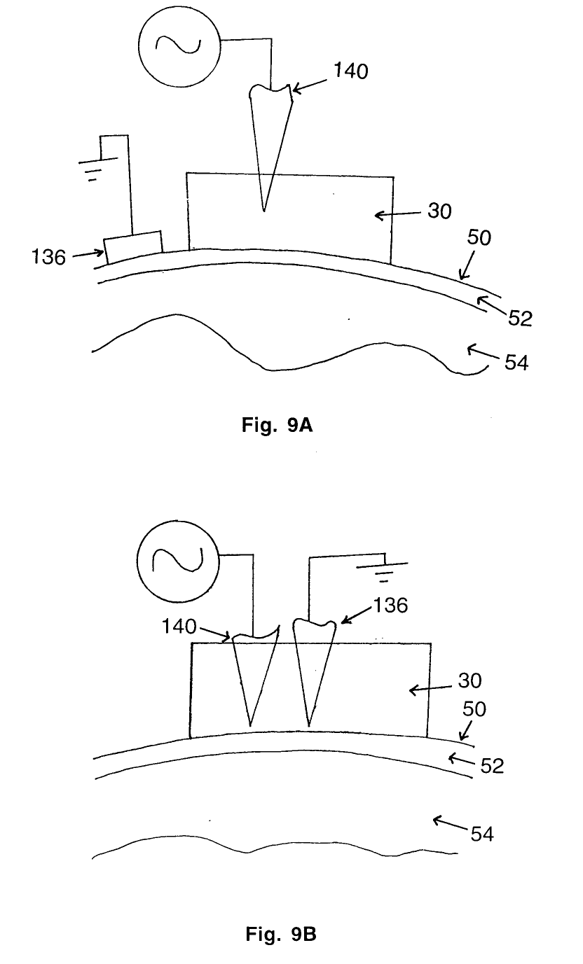

FIG. 9A depicts an active electrode 140 in contact with the fusion composition 30 which is placed on the stratum corneum 52 of the skin 50. A ground electrode 135 is located distal to the active electrode 140 and the fusion composition 30 and also is in contact with the stratum corneum 52. A plasma (not shown) forms, upon the application of radiofrequency energy or direct current, between the electrodes 140, 135 alters the stratum corneum without harming the epidermis 54 underneath the stratum corneum 52. Additionally, beneficial thermal changes are created within the fusion composition 30. Alternatively, FIG. 9B places both the active electrode 140 and the ground electrode 135 within the fusion composition 30.

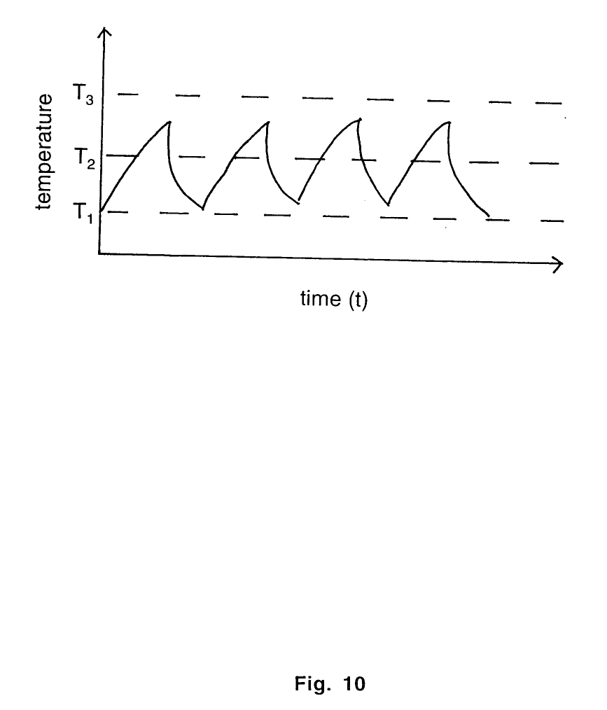

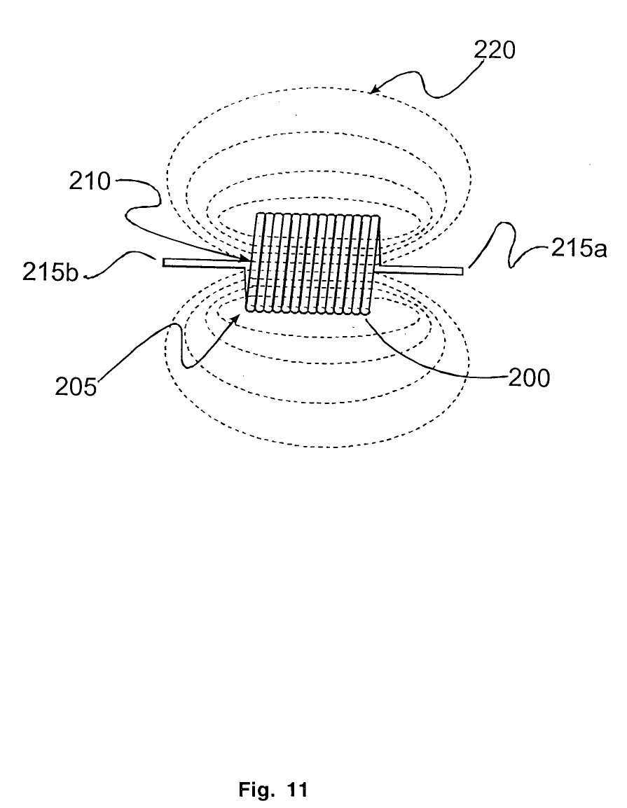

FIG. 10 depicts an example of a heating history of a fusion compound whereby T1 is the local body temperature, T2 is the peak temperature required for cure of the fusion compound, and T3 is the temperature at which deleterious thermal damage may occur in the tissue. The example heating history is sufficient to cause the desired change in the fusion compound, without causing undesirable thermal damage.

FIG. 11 depicts an applicator 205 having an essentially solenoid structure 200 which is formed with an interior cylindrical zone 210. The magnetic field lines 220 produced when an electrical current is passed through the wire at 215a,b is shown. While the greatest magnetic intensity H (A/m) occurs within the applicator, a weaker magnetic field occurs at the ends and outside of the solenoid.

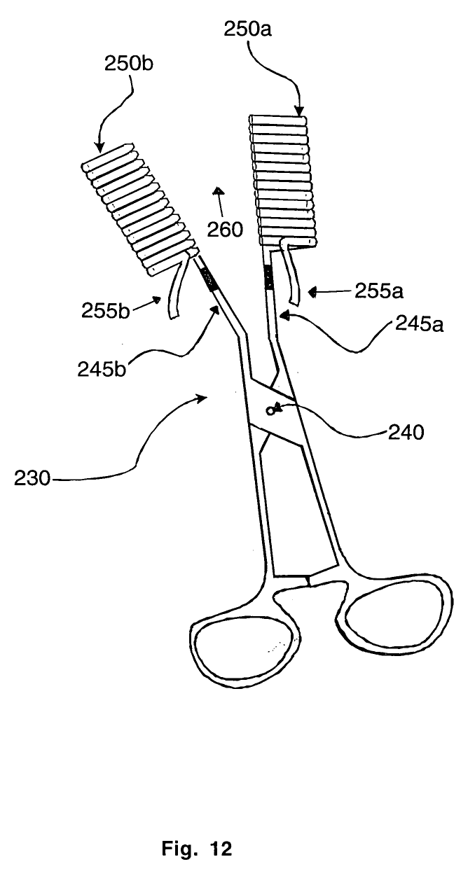

In FIG. 12 and with continued reference to FIG. 11, a solenoid-type applicator 230 is constructed such that the coil-halves of the solenoid 250a,b can be opened, closed or adjusted via a clamp-like handle 240, thus allowing the positioning of an anatomical structure within the interior cylindrical zone 260. When the coil-halves 250a,b are closed via the scissors-like action of the handle 240, electrical contact is established. The resulting intensity H is consistent with the field 220 shown in FIG. 11. The handle 240 is electrically isolated from the coil-halves 250a,b by insulators 245a,b. The power is conducted to the coil with electrical leads 255a,b.

FIGS. 13A-13C depict substantially flat applicator coils for activating in other anatomical geometries. FIG. 13A is a “butterfly coil” 270 with electrical connectors 271a,b. FIG. 13B is a spiral coil 274 with electrical connectors 273a,b. FIG. 13C is an alternative spiral coil 278 with electrical connectors 279a,b. Each coil produces a magnetic field with a particular geometric shape. For example, coil 270 produces a two-lobed shaped field above and below the flat plane of the coil. With the addition of a material, such as mu-metal (not shown), it is possible to shield the superior surface of the coil 270 if no magnetic field is desired above the coil.

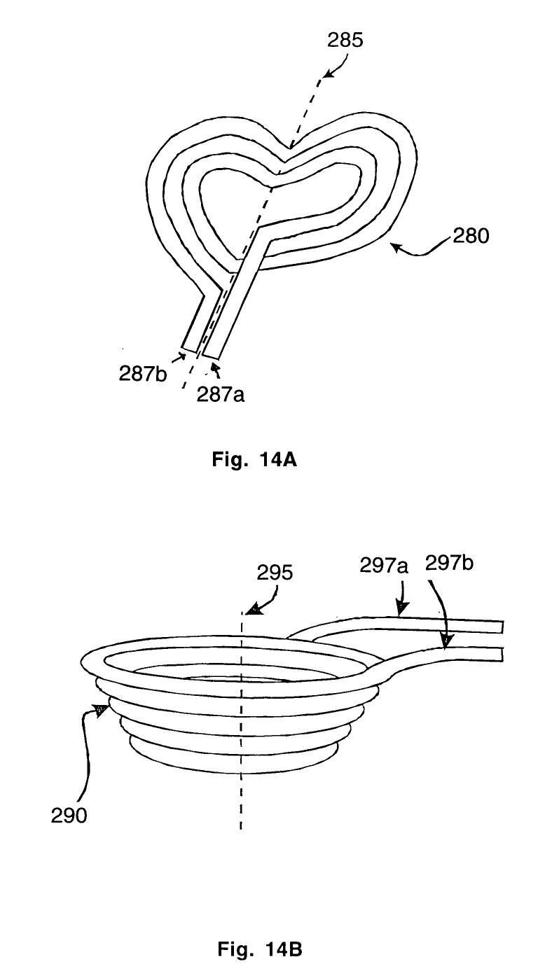

In FIGS. 14A-14B and with continued reference to FIG. 13, non-planar coil applicators are illustrated. FIG. 14A depicts a coil 280 similar to 274 in FIG. 13, however each half of 280, as delineated by a centerline 285 is bent towards and along the centerline 285, thus increasing the magnetic field intensity H at a position within a volume contained within the bent coil 280. The power is connected to the coil through leads 287a,b. FIG. 14B depicts a coil 290 which is in the form of a conical spiral with axis of symmetry 295. The power is connected to the coil through leads 297a,b.

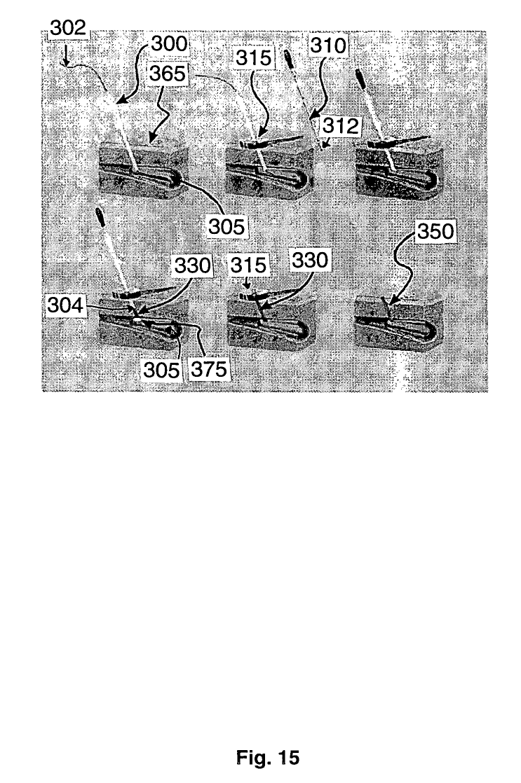

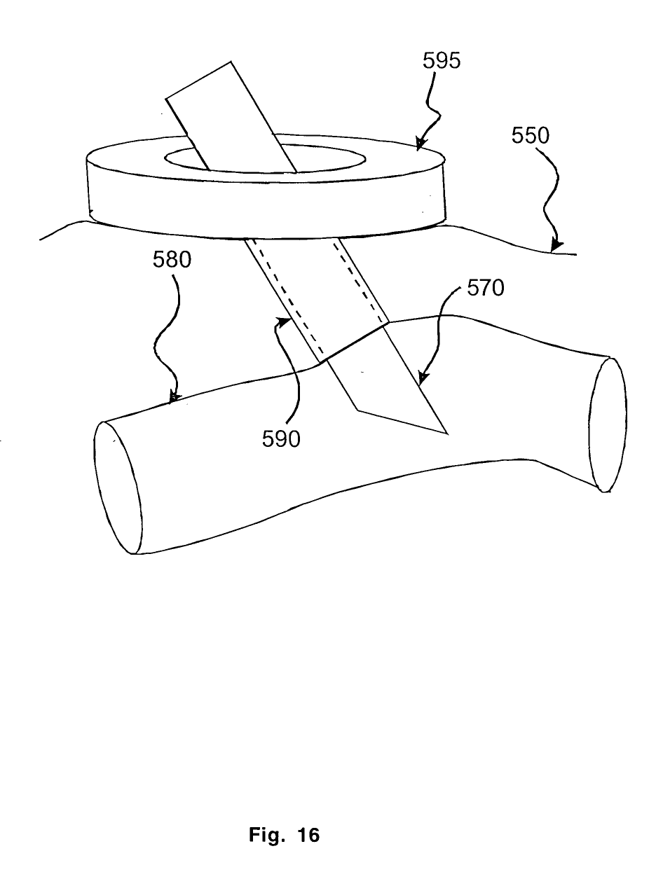

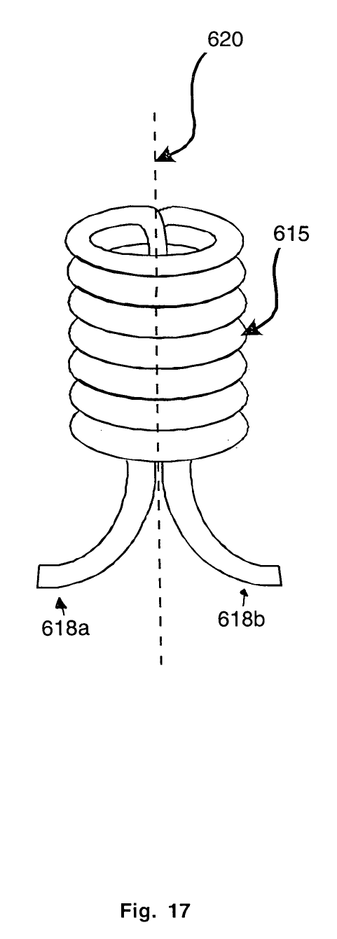

FIG. 15 depicts an applicator and its method of use for closing a vascular access defect in tissue. A catheter introducer 300 is used to allow access to the lumen 305 of a blood vessel within a tissue 365. After the catheterization procedure and removal of the guidewire 302, a tissue-fusion applicator 315 is positioned in proximity to the introducer 300. A fusion composition delivery device 310 is placed within the lumen of the introducer 300.

A material 375 is located within the distal end 312 of the fusion composition delivery device. A small amount of material 375 is extruded out the distal end 304 of the introducer 300 with the delivery device 310 into the lumen 305 of the blood vessel to provide accurate positioning of the composition delivery device 310 and to temporarily occlude the vascular perforation. The material 375 is biocompatible and dissolves in the blood stream within minutes or hours of the procedure. Fusion composition 330 contained within the applicator 315 is delivered to the vascular access defect as the fusion composition delivery device 310 and the introducer 300 are withdrawn. The fusion composition 330 remaining in the defect is activated by the applicator 315 thus sealing the puncture 350 in the blood vessel and skin.

FIG. 16 depicts an applicator suitable for closing a needle tract defect in tissue. A sheath 590 composed of fusion composition surrounds a needle 570 which is positioned within a blood vessel 580 within tissue 550 for the purpose of venipuncture. The tissue fusion applicator 595 is positioned close to the needle 570 and sheath 590. Upon withdrawal of the needle 570, the sheath 590 collapses, or is filled with fusion composition, and is activated with the fusion applicator 595 to substantially seal the needle tract.

FIG. 17 depicts an applicator coil 615 which is symmetric around axis 620. The power is conducted to the coil 615 with electrical leads 618a,b. The applicator is designed for use in a hollow anatomical structure, such as a blood vessel.

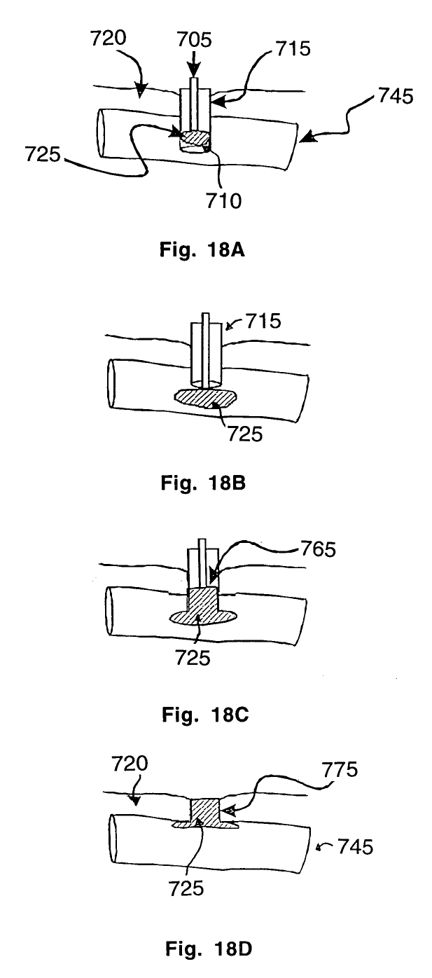

FIGS. 18A-18D depict an applicator suitable for closing a vascular access defect or other defect in tissue. In FIG. 18A an introducer 715 is positioned in the tissue 720 and perforates a vessel 745. Within the lumen of the introducer 715 is a plunger 705 and fusion composition 725. Here the fusion composition 725 swells upon contact with blood. In FIGS. 18B-C the fusion composition 725 is extruded out the end 710 of the introducer 715 whereupon it expands. In FIG. 18C, upon withdrawal of the introducer 715 and retraction of the plunger 705, the bond 765 between the fusion composition 725 and plunger 705 can be breached. In FIG. 18D the fusion composition 725 fills the vascular perforation in the vessel 745 and tissue defect in the tissue 720. If required, the fusion composition 725 can be activated with an externally positioned applicator such as shown in FIG. 16.

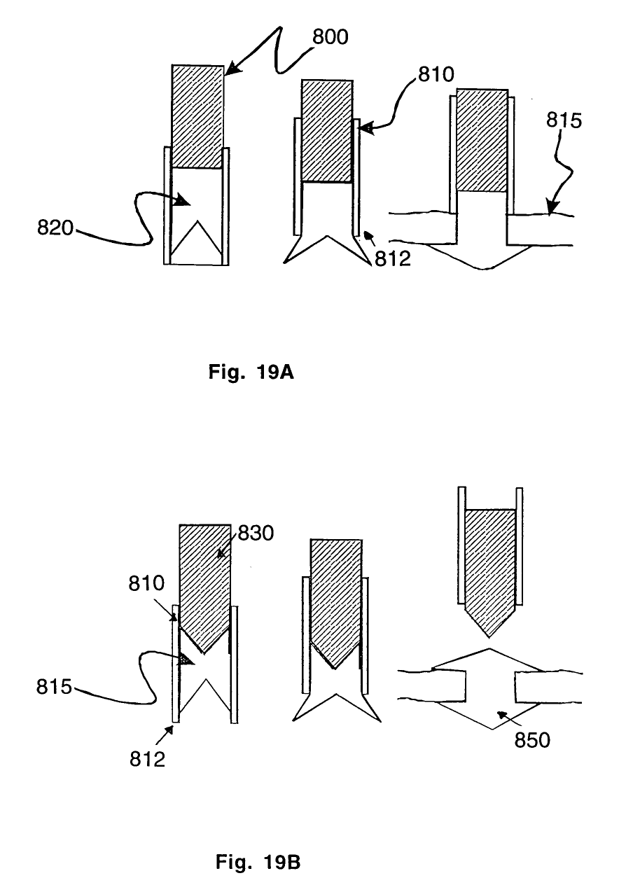

FIGS. 19A-B depict examples of self-expanding fusion compositions. In FIG. 19A, a plunger 800 is used to push a fusion composition 820 out the end 812 of a sheath 810 positioned in tissue 815 such as a blood vessel. Upon retraction of the sheath 810 and plunger 800, the composition 820 expands in a way to seal the defect in the tissue 815. In FIG. 19b, another fusion composition 830 is depicted which serves to occlude the defect in tissue 815 from both sides. The fusion composition 850 is pushed out the end 812 of the sheath 810 with plunger 830. Upon retraction of the sheath 810 and plunger 830, the composition 850 expands to seal the defect in the tissue 815.

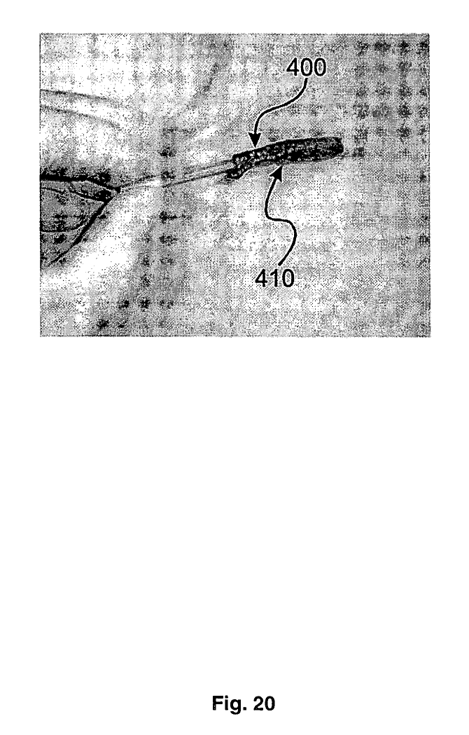

FIG. 20 depicts the visible fusion 410 of a vascular vessel 400.

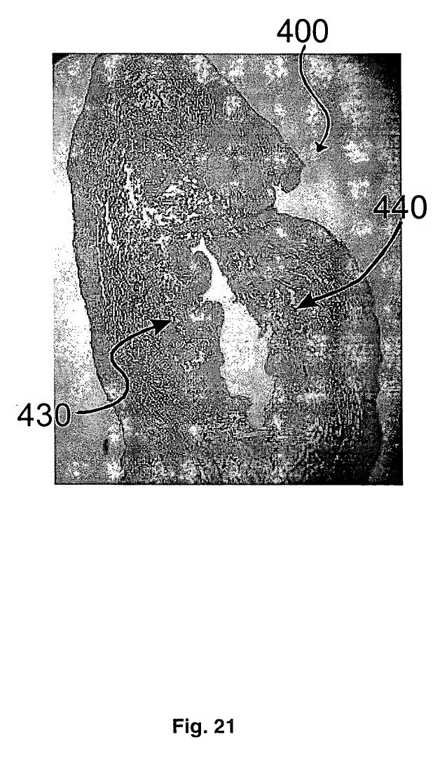

FIG. 21, with reference to FIG. 20, shows a histological section of the vascular vessel 400 with metallic particles 430 and 440 at the interface 410 between the two overlapping sections.

The following examples are given for the purpose of illustrating various embodiments of the invention and are not meant to limit the present invention in any fashion.

### EXAMPLE 1

Heating of Test Metal

The prototype activator device was constructed (Quest Product Development, Evergeen, Colo.) and operates at a frequency of about 650 kHz with an output of approximately 210 W. At or near this frequency, the skin depth in tissue, e.g., canine skeletal muscle at 1 MHz, (Francis Duck. Physical Properties of Tissue—A comprehensive reference book. Academic Press: NY, 1990) is about 205 cm and for nickel it is about 14 microns. Thus, no significant heating of tissue occurs as a direct result of the field. Heating only occurs in close proximity to the fusion composition.

Two applicator designs were used and comprised 200 turns of solid copper wire, 32 and 22 G, resulting in a coil approximately 2.86 cm in diameter and 0.95 cm in width. The bore of the coil was about 0.5 cm. The coils were encapsulated in a Pyrex sleeve through which low-viscosity mineral oil (Sigma-Aldrich Inc., St. Louis, Mo.) is circulated as a coolant. In each of these coils, the magnetic intensity at the center of the coil is calculated to be greater than 10,000 A/m, while at approximately 0.5 cm from a single coil face the intensity is calculated to be maximally 160 A/m. The fusion composition was bovine serum albumin, 25, 50 and 75% by weight in water, combined with 325 mesh nickel flake (Alfa Aesar, Ward Hill, Mass.). The composition was homogeneously mixed and used immediately afterwards.

Aliquots of approximately 1 ml of the fusion composition were positioned in thin-walled glass tubes with a diameter of about 4 mm. The tube was then positioned in the bore of the applicator. The device was energized for a period of 30 seconds. Evidence of denaturation and coagulation was ascertained visually as the material changed color. This was confirmed by probing the composition with a needle and looking for evidence of increased viscosity or stiffness. The composition coagulated with all combinations of applicator and compositions.

A small screwdriver (Craftsman Model 41541, 3.15 mm diameter) was positioned within the bore of the coils. After 1-5 seconds, the screwdriver was extracted and the metal was brought transiently into contact with the skin of the hand. It was immediately apparent that significant heating had taken place.

### EXAMPLE 2

Heating and Coagulating of Test Fusion Formulation

Fusion formulations were made of 50-75% (w/v) bovine serum albumin or ovalbumin (Sigma-Aldrich, St. Louis, Mo.) in saline with a metal additive of 5% or 10% (w/v) nickel flake with average particle size of about 50 micron (Alfa Aesar, Ward Hill, Mass.) or 10% iron filings with particle size <30 microns (Edmund Scientific, Tonawanda, N.Y.). Approximately 1 ml aliquots of the fusion composition were positioned in thin-walled glass tubes with a diameter of about 4 mm. The tube was positioned in the bore of the applicator. The device was energized for a period of 20-30 seconds. Evidence of denaturation and coagulation was ascertained visually, as the material changed color. This was confirmed by probing the composition with a needle which demonstrated evidence of increased viscosity or stiffness. The composition coagulated with all combinations of applicator and composition. Compositions with more metal or iron versus nickel heated at different rates.

### EXAMPLE 3

Protein Denaturation

A radiofrequency electromagnetic device, operating at 650 kHz, was constructed. Near this frequency, the skin depth in tissue, using conductivity values for canine skeletal muscle at 1 MHz, is about 205 cm, while for nickel, it is 14 μm. Two solenoid type coils were constructed using 20 G solid copper wire. The coils were encapsulated in a Pyrex sleeve through which low-viscosity mineral oil is circulated as a coolant. Two coils had 200 turns of solid copper wire, formed into a solenoid, with a diameter of 2.86 cm and width of 0.95 cm. The magnetic intensity within the bore of the coil was calculated to be greater than 100 kA/m, while at approximately 0.5 cm from a single coil face the intensity is calculated to be maximally 0.15 kA/m. Two coils were electronically connected to the radiofrequency power supply and physically arranged with the bore axis parallel and opposing each other with a gap of about 2 cm between the faces of the coils.

The reactant was ovalbumin at a concentration of 50% (w/v) albumin in 0.9% saline as a high viscosity liquid) or 75% (w/v) albumin as a paste. The transducer species was nickel flake with an average particle size of about 46 micron, mixed into the albumin solution at 5-10% (w/v). The mixture of albumin, saline and nickel had a highly viscous rheological nature. The fusion composition preparation showed visual evidence through coagulation and change in opacity and was warm to the touch after 20-30 seconds when placed between the two solenoid coils with the radiofrequency power supply producing about 210 W.

### EXAMPLE 5

Tissue Fusion

Ex vivo sheep arteries were dissected transversely across the lumen to form sections or were cut longitudinally to form sheets of tissue. The fusion composition was sandwiched between small sections, i.e., about 1 cm², of the tissue sheets and was placed between the coils as before. Tissue fusion was apparent by observation. The tissues fused together seamlessly and it became difficult to tease apart the two sections with forceps. No effort was made to control temperature, however, overheating was apparent from a color change in the tissue with longer exposure times of >45 seconds.

Next one fusion composition, 5% Ni, 50% albumin, was placed on the adventitia of one end of a transverse-cut vessel and the end of another dissected vessel was placed over the adventitia and a 200 micron layer of the adhesive. A glass rod was used as a support to hold the tissue in place. The sample was then positioned between the faces of the opposing coils and the sample was exposed for about 30 seconds.

The magnetic intensity between the two coils theoretically is estimated to be about 0.3 kA/m. Fusion was visually apparent (FIG. 20) after about 90 seconds and the fused tissue could not be teased apart with forceps without dissecting the tissue. Tests were repeated five times with equivalent results. The vessels were placed in 10% formalin, sectioned transversely across the fused area and submitted for histological preparation and staining with hematoxylin-eosin. The presence of metallic transducer particles was apparent (FIG. 21) at the interface between the two overlapping sections and delineates the margin of tissue fusion.

### EXAMPLE 8

Effects of Inductive Heating on Fusion Compositions

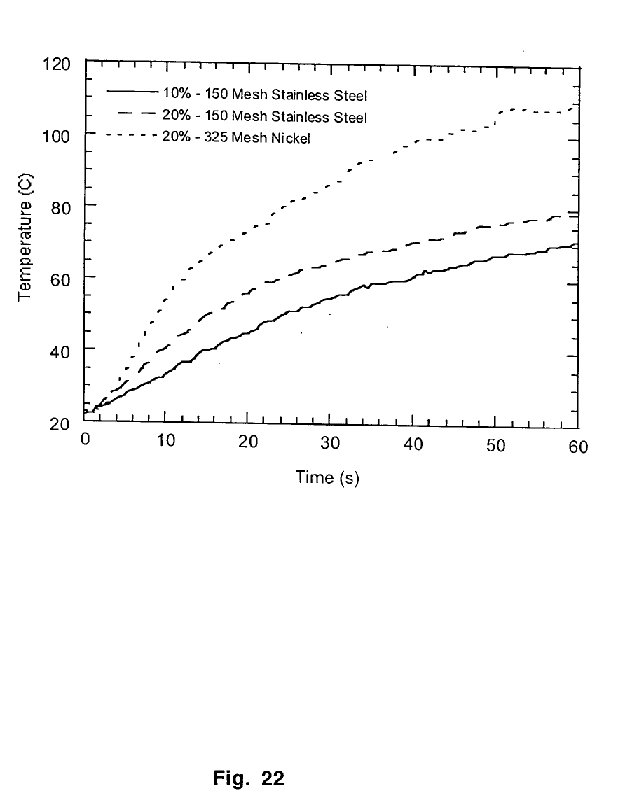

A commercially available induction power-supply (Lepel Corp., Edgewood, N.Y.) modified through the addition of internal capacitors to accept a solenoid coil was used. The device produced an average power of about 100 W at a frequency of 400 kHz and a field intensity of 0.3 A/m. The output of the device was coupled into a helical wound coil with an outside diameter of 11 cm made of 11 turns of ⅛ inch copper tubing. The fusion compositions tested contained 50% albumin with a tranducer consisting of 10% 150 mesh stainless steel or 20% 150 mesh stainless steel or 20% 325 mesh nickel. Each fusion composition was separately positioned within the bore of the coil flush with the surface, and the temperature of the upper surface of the fusion composition was measured with an infrared thermometer. The results from heating three different fusion compositions for 60 seconds is shown in FIG. 22. As expected, nickel heats more efficiently than stainless steel due to its greater magnetic permeability, reaching a threshold temperature of −70° C. within 30 seconds, while stainless steel transducers require double the time.

The following references are cited herein:

1. Bass L S and Treat M R. Laser Surg. Med. 17, 315-349, (1995).

2. Freid N M and Walsh J T. Lasers Surg. Med. 27, 55-65, (2000).

3. Davies E J. Conduction and Induction Heating. Inst. Elect. Engs. and P. Peregrinus: London (1990).

4. Orfeuil, M. Electric Process Heating: Technologies/Equipment/Applications. Battelle Press: Columbus Ohio (1987).

5. Zinn S. and Semiatin S L. Elements of Induction Heating—Design, Control and Applications, Electric Power Research Institute: Palo Alto, Calif. (1988).

6. Damodaran S. Influence of protein conformation on its adaptability under chaotropic conditions. Int. J. Biologic. Macromolec. 11, pp.2-8, (1989).

7. Stauffer P R, Cetas R C and Jones R C. IEEE Trans. Biomed. Eng. BME-31, 235-251 (1984).

8. Hamad-Schifferli K, Schwartz J J, Santos A T, Zhang S and Jacobson J M. Nature 415, 152-155 (2002).

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method for heating one or more substrates, comprising:
applying electromagnetic energy to an antenna to generate an electromagnetic field therefrom; and
heating said substrate(s) via said electromagnetic field.

2. The method of claim 1, further comprising:
heating an energy absorbing species with a non-zero electrical conductivity via said electromagnetic field prior to delivering heat energy to said substrate(s), said absorbing species delivering said heat energy to said substrate(s).

3. The method of claim 2, wherein said energy absorbing species is diamagnetic, paramagnetic or ferromagnetic.

4. The method of claim 2, wherein said energy absorbing species is an ionomer, a conducting polymer, an alkali metal, a transition metal, a lanthanide, or a metalloid or a combination thereof.

5. The method of claim 2, wherein said energy absorbing species is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

6. The method of claim 2, wherein said energy absorbing species is a metal nano- or micro-particle, a semiconducting nano- or micro-particle, a magnetic nano- or micro-particle, a polystyrene encapsulated metal particle, a buckminsterfullerene, or a liposome-encapsulated metal particle.

7. The method of claim 1, wherein at least one of said substrates is a tissue, a cell, a biomolecule, a biologically active molecule, an adhesive, or a combination thereof.

8. The method of claim 7, wherein said biomolecule or said biologically active molecule is a protein, a lipid, a nucleic acid, or a carbohydrate.

9. The method of claim 7, wherein the biomolecule or biologically active molecule is a pharmaceutical, a biologic, a biomaterial, a diagnostic, or a biological marker.

10. The method is claim 1, wherein the electromagnetic energy is radiofrequency energy.

11. The method of claim 10, wherein said radiofrequency energy has a frequency of about 20 KHz to about 40 GHz.

12. The method of claim 1, wherein the electromagnetic energy generates a magnetic field.

13. The method of claim 1, wherein said antenna comprises at least one coil of electrical conductor.

14. The method of claim 13, wherein said electrical conductor is a solid wire or hollow tubing.

15. The method of claim 1, wherein said antenna is a single coil antenna, a double coil antenna or a solenoid antenna.

16. The method of claim 1, wherein heating said substrates(s) cleaves a bond in at least one of said substrate(s), denatures at least one of said substrate(s) or shrinks at least one of said substrate(s).

17. A method of inducing an alteration in a substrate, comprising:
delivering energy to an energy absorbing species;
heating said energy absorbing species via said energy; and
transferring heat energy from said energy absorbing species to heat said substrate thereby inducing the alteration therein.

18. The method of claim 17, wherein said energy absorbing species is a susceptor.

19. The method of claim 17, wherein said energy absorbing species comprises matter with non-zero electrical conductivity.

20. The method of claim 17, wherein said energy absorbing species is diamagnetic, paramagnetic, or ferromagnetic.

21. The method of claim 17, wherein said energy absorbing species is an ionomer, a conducting polymer, an alkali metal, a transition metal, a lanthanide, or a metalloid or a combination thereof.

22. The method of claim 21, wherein said energy absorbing species is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

23. The method of claim 17, wherein said energy absorbing species is a metal nano- or micro-particle, a semiconducting nano- or micro-particle, a magnetic nano- or micro-particle, a polystyrene encapsulated metal particle, a buckminsterfullerene, or a liposome-encapsulated metal particle.

24. The method of claim 17, wherein said energy is radiofrequency energy.

25. The method of claim 24, wherein said radiofrequency energy has a frequency of about 20 KHz to about 40 GHz.

26. The method of claim 17, wherein the energy generates an electromagnetic field.

27. The method of claim 26, wherein said electromagnetic field is generated via an antenna.

28. The method of claim 27, wherein said antenna comprises at least one coil of electrical conductor.

29. The method of claim 28, wherein said electrical conductor is a solid wire or hollow tubing.

30. The method of claim 28, wherein said antenna is a single coil antenna, a double coil antenna or a solenoid antenna.

31. The method of claim 17, wherein the substrate is a biomolecule, a biologically active molecule, a tissue, or a cell.

32. The method of claim 31, wherein said biomolecule or biologically active molecule is a protein, a lipid, a nucleic acid, or a carbohydrate.

33. The method of claim 31, wherein said biomolecule or biologically active molecule is a pharmaceutical, a biologic, a biomaterial, a diagnostic, or a biological marker.

34. The method of claim 17, wherein said substrate is inorganic.

35. The method of claim 34, wherein said substrate is a shape memory alloy.

36. The method of claim 17, wherein the alteration induced in said substrate is a cleaved bond, is denaturation of said substrate or is contraction of said substrate.

37. A device for the treatment of substrates, comprising:
a radiofrequency power supply;
an energy absorbing species; and
a means for inductively applying said radiofrequency energy to said substrates.

38. The device of claim 37, wherein the substrates are biologicals, biologically active materials, tissues, or cells.

39. The device of claim 37, wherein said substrates are in vitro.

40. The device of claim 37, wherein the power supply generates radiofrequency energy from about 20 KHz to about 40 GHz.

41. The device of claim 37, wherein said means of inductively applying energy comprises an antenna.

42. The device of claim 41, wherein said antenna comprises at least one coil of electrical conductor.

43. The device of claim 42, wherein said electrical conductor is solid wire or hollow tubing.

44. The device of claim 41, wherein said antenna is a single coil antenna, a double coil antenna or a solenoid antenna.

45. The device of claim 37, wherein said energy absorbing species is a susceptor.

46. The device of claim 37, wherein said energy absorbing species comprises matter with non-zero electrical conductivity.

47. The device of claim 37, wherein said energy absorbing species is diamagnetic, paramagnetic, or ferromagnetic.

48. The device of claim 37, wherein said energy absorbing species is an ionomer, a conducting polymer, an alkali metal, a transition metal, a lanthanide, or a metalloid or a combination thereof.

49. The device of claim 48, wherein said energy absorbing species is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

50. The device of claim 37, wherein said energy absorbing species is a metal nano- or micro-particle, a semiconducting nano- or micro-particle, a magnetic nano- or micro-particle, a polystyrene encapsulated metal particle, a buckminsterfullerene, or a liposome-encapsulated metal particle.

51. A composition used in the treatment of one or more substrates, comprising:
at least one reactant; and,
at least one energy absorbing species.

52. The composition of claim 51, wherein said reactant(s) is a protein, a lipid, a nucleic acid, or a carbohydrate.

53. The composition of claim 51, wherein said reactant(s) comprises a pharmaceutical, a biologic, a biologically active molecule, a diagnostic, a biological marker or a combination thereof.

54. The composition of claim 51, wherein said energy absorbing species comprises matter with non-zero electrical conductivity.

55. The composition of claim 51, wherein said energy absorbing species is diamagnetic, paramagnetic, or ferromagnetic.

56. The composition of claim 51, wherein said energy absorbing species is an ionomer, a conducting polymer, an alkali metal, a transition metal, a lanthanide, or a metalloid or a combination thereof.

57. The composition of claim 56, wherein said energy absorbing species is colloidal or non-colloidal gold, silicon, cadmium selenide, cadmium sulfide, ruthenium, indium phosphide, indium arsenide, gallium arsenide, gold maleimide, gallium phosphide, hydroxysuccinimidyl gold, nickel-copper, nickel-palladium, palladium-cobalt, nickel-silicon, stainless steel, iron oxide, ferrite, titanium, Phynox, palladium/cobalt alloys, nitinol, titanium, titanium alloys, zirconium, gadolinium, aluminum oxide, dysprosium, cobalt alloys, nickel, gold, palladium, tungsten, or alloys of materials from this group.

58. The composition of claim 51, wherein said energy absorbing species is a metal nano- or micro-particle, a semiconducting nano- or micro-particle, a magnetic nano- or micro-particle, a polystyrene encapsulated metal particle, a buckminsterfullerene, or a liposome-encapsulated metal particle.

59. A method of treatment for one or more substrates in an individual, comprising:
positioning the substrates;
applying the composition of claim 51 to at least one of said substrates;
applying energy to said composition; and
curing said composition thereby treating said substrate(s).

60. The method of claim 49, wherein said substrate(s) is a biomolecule, a biologically active molecule, a tissue. or a cell.

61. The method of claim 59, wherein at least one of said substrates is an implant or a bandage.

62. The method of claim 59, wherein curing said composition seals said substrate(s), fills-in an opening in said substrates, fuses said substrate(s), or fixes said substrate(s).

63. The method of claim 59, wherein curing said composition denatures at least one of said reactants or changes molecular structure of at least one of said reactants.

64. The method of claim 59, wherein said energy is radiofrequency energy, magnetic or an electrical current.

65. The method of claim 64, wherein said radiofrequency energy has a frequency from about 20 KHz to about 40 GHz.

66. The method of claim 59, wherein said energy is applied via an antenna.

67. The method of claim 66, wherein said antenna comprises at least one coil of electrical conductor.

68. The method of claim 67, wherein said electrical conductor is solid wire or hollow tubing.

69. The method of claim 66, wherein said antenna is a single coil antenna, a double coil antenna or a solenoid antenna.
